(19) United States
(12) Patent Application Publication
(10) Pub. No.: US 2022/0079953 A1
(43) Pub. Date: Mar. 17, 2022

(54) METHODS FOR MANAGEMENT OF WEIGHT

(71) Applicants: The University of Sydney, New South Wales (AU); Macquarie University, North Ryde, New South Wales (AU)

(72) Inventors: Iain Stewart MCGREGOR, New South Wales (AU); Michael Thomas BOWEN, New South Wales (AU); Sarah J. BARACZ, North Ryde, New South Wales (AU); Jennifer L. CORNISH, North Ryde, New South Wales (AU)

(21) Appl. No.: 17/295,638

(22) PCT Filed: Nov. 22, 2019
(86) PCT No.: PCT/AU2019/051284

§ 371 Date: May 20, 2021

(30) Foreign Application Priority Data
Nov. 23, 2018 (AU) 2018904479

(51) Int. Cl. A61K 31/5517; A61K 31/553; A61K 31/554; A61P 3/04; A61K 31/4745

(52) U.S. Cl. CPC A61K 31/5517; A61K 31/553; A61K 31/4745; A61P 3/04; A61K 31/554

(57) ABSTRACT

Disclosed herein are methods for causing weight loss, for managing weight, and for suppressing an appetite for food in a subject, and methods for reducing the consumption of food by a subject by administering the compounds, or pharmaceutically acceptable salts or prodrugs thereof, of the invention.

### FIELD OF THE INVENTION

The present invention relates to the use of novel compounds in the management of weight.

### RELATED APPLICATION

This application claims priority from Australian provisional application AU2018904479, the entire contents of which are hereby incorporated by reference.

### BACKGROUND OF THE INVENTION

Excess weight and obesity is a major public health issue and among the leading risk factors for cardiovascular disease, type 2 diabetes, some musculoskeletal conditions and some cancers. As the level of excess weight increases, so does the risk of developing these conditions. In addition, being overweight or obese can hamper the ability to control or manage chronic conditions. An unhealthy diet (particularly a high sugar and/or high fat diet) is considered a primary determinant of excess weight and obesity.

Current pharmacological treatments for overweight and obese individuals do not provide clinically meaningful benefits for about 30 to 65% of patients (depending on the treatment and study examined), and most current drugs are marred by considerable side effects.

Therefore, there is a need for new treatments that can improve the outcomes for overweight individuals, as well as prevent those who may be predisposed to consuming excessive amounts of food, from becoming overweight or obese.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In a first aspect, the present invention relates to a method for causing weight loss in a subject, the method including a step of administering to the subject a compound of Formula (I), or a pharmaceutically acceptable salt, tautomer, N-oxide, solvate, polymorph and/or prodrug thereof:

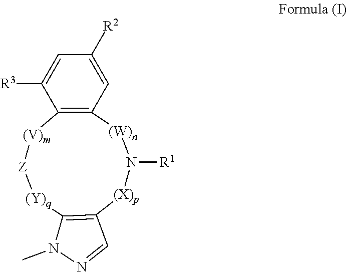

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

In a second aspect, the present invention relates to a method for managing weight in a subject, the method including a step of administering, to the subject, a compound of Formula (I), or a pharmaceutically acceptable salt, tautomer, N-oxide, solvate, polymorph and/or prodrug thereof, as described in the first aspect of the invention.

In a third aspect, the present invention relates to a method for suppressing an appetite for food in a subject, the method including a step of administering to the subject a compound of Formula (I), or a pharmaceutically acceptable salt, tautomer, N-oxide, solvate, polymorph and/or prodrug thereof, as described in the first aspect of the invention. The appetite for food may be an uncontrolled appetite for food.

In a fourth aspect, the present invention relates to a method for controlling, reducing or minimising food intake by a subject, the method including a step of administering to the subject a compound of Formula (I), or a pharmaceutically acceptable salt, tautomer, N-oxide, solvate, polymorph and/or prodrug thereof, as described in the first aspect of the invention.

The subject may be at risk of becoming overweight. The subject may be overweight. The subject may be obese. The subject may have a disorder that leads to weight gain through excessive consumption of food, such as a disorder selected from binge eating disorder, subclinical binge eating, anxiety, depression, Prader-Willi syndrome and metabolic syndrome (also known as syndrome X or insulin-resistance syndrome). The subject may have other excess weight-related metabolic disorders. Examples of these include insulin resistance, glucose intolerance, pre-diabetes, increased fasting glucose, type 2 diabetes, hypertension, dyslipidemia (or a combination of these metabolic risk factors), glucagonomas, cardiovascular diseases (such as congestive heart failure, atherosclerosis, arteriosclerosis, coronary heart disease, or peripheral artery disease), stroke, respiratory dysfunction, or renal disease.

The food may be high-fat, high-carbohydrate and/or high sugar food.

The compound may be:

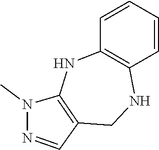

or a pharmaceutically acceptable salt, tautomer, N-oxide, solvate, polymorph and/or prodrug thereof.

The present invention also provides a use of a compound of Formula (I), or a pharmaceutically acceptable salt, tautomer, N-oxide, solvate, polymorph and/or prodrug thereof, in the manufacture of a medicament for:
causing weight loss in a subject;
managing weight in a subject:
suppressing an appetite for food in a subject, including an uncontrolled appetite for food; or
controlling, reducing or minimising food intake by a subject.

Further still, the present invention provides a compound of Formula (I), or a pharmaceutically acceptable salt, tautomer, N-oxide, solvate, polymorph and/or prodrug thereof, for use in:
causing weight loss in a subject;
managing weight in a subject:
suppressing an appetite for food in a subject, including an uncontrolled appetite for food; or
controlling, reducing or minimising food intake by a subject.

In any method or use described herein, the food for which the appetite is suppressed, or for which intake is controlled, reduced or minimised is selected from the group consisting of: a high-fat food, a high-sugar food. In further embodiments, the high-sugar food is a high-caloric drink (including a soft drink, fruit juice, fruit purée, formulated beverage).

As used herein, except where the context requires otherwise, the term “comprise” and variations of the term, such as “comprising”, “comprises” and “comprised”, are not intended to exclude further additives, components, integers or steps.

The present invention also provides a kit for use in accordance with the methods of the present invention, optionally wherein the kit includes written instructions for performing the methods of the invention.

In any method, use or kit of the invention, the compound of Formula (I) is:

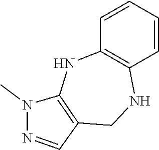

or a pharmaceutically acceptable salt, tautomer, N-oxide, solvate, polymorph and/or prodrug thereof.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Effect of SOC-1 on lever presses under a progressive ratio to receive either one or five M&Ms per reinforcement in rhesus monkeys (* p<0.05 vs 0 mg/kg SOC-1).

FIG. 2. Effect of SOC-1 on lever presses to self-administer sucrose under an FR5 reinforcement schedule in rats (***p<0.001 vs 0 mg/kg SOC-1).

FIG. 3. Extinguishment of response to sucrose and effect of SOC-1 on reinstatement of sucrose-seeking in rats (*** p<0.001 vs extinction; p<0.001 vs 0 mg/kg reinstatement).

FIG. 4. Effect of SOC-1 on extinction of non-reinforced responding for food in food-deprived rats (** p<0.01,*** p<0.001, **** p<0.0001 vs VEH).

FIG. 5. Effect of SOC-1 dosed once daily for a ten day period in male Sprague-Dawley rats with ad libitum access to standard chow. n=5 per dose group, N=20. Significant difference from 0 mg/kg group (***p<0.001, ****p<0.0001) in ANOVA with Dunnet's multiple comparison test.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

For purposes of interpreting this specification, terms used in the singular will also include the plural and vice versa.

The present inventors have surprisingly found that compounds of Formula (I) (shown below) are effective at reducing consumption of food, particularly types of food that are known to cause weight gain and obesity.

Accordingly, the present invention provides a method of causing weight loss in a subject, the method including a step of administering to the subject a compound of Formula (I), or a pharmaceutically acceptable salt, tautomer, N-oxide, solvate, polymorph and/or prodrug thereof:

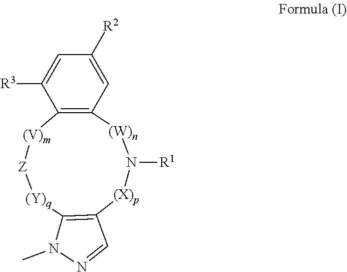

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

As used herein, the term “C₁₋₅ alkyl” either used alone or in compound terms, refers to monovalent straight chain or branched hydrocarbon groups, having 1 to 5 carbon atoms. As understood by a person skilled in the art, the term “C₁₋₅ alkyl” means an alkyl chain with 1, 2, 3, 4 or 5 carbon atoms or a range including any of two of those integers including 1-2, 1-3, 1-4, 1-5, 2-3, 2-4, 2-5, 3-4, 3-5 and 4-5. Suitable alkyl groups include, but are not limited to: methyl, ethyl, propyl, isopropyl, n-butyl, sec-butyl, tert-butyl, n-pentyl, neopentyl, iso-pentyl and tert-pentyl. The C₁₋₄ alkyl may be optionally substituted with one or more substituents. The substituents may be in any position of the carbon chain. Suitable substituents include, but are not limited to: OH, NH₂, halogen, NH(C₁₋₅ alkyl), N(C₁₋₅ alkyl)₂, CN, NO₂, CO₂H, or OC₁₋₅ alkyl.

As used herein, the term “OC₁₋₅ alkyl” either used alone or in compound terms, refers to alkoxy groups having 1 to 5 carbon atoms. As understood by a person skilled in the art, the term “OC₁₋₅ alkyl” means an alkoxy group with 1, 2, 3, 4 or 5 carbon atoms or a range including any of two of those integers and including 1-2, 1-3, 1-4, 1-5, 2-3, 2-4, 2-5, 3-4, 3-5 and 4-5. Suitable OC₁₋₅ alkyl groups include, but are not limited to, methoxy, ethoxy, propyloxy, isopropyloxy, n-butyloxy, sec-butyloxy, tert-butyloxy, n-pentyloxy, neopentyloxy, iso-pentyloxy and tert-pentyloxy. The C₁₋₅ alkyl may be optionally substituted with one or more substituents. The substituents may be in any position of the carbon chain. Suitable substituents include, but are not limited to: OH, NH₂, halogen, NH(C₁₋₅ alkyl), N(C₁₋₅ alkyl)₂, CN, NO₂, CO₂H, or OC₁₋₅alkyl.

As used herein, the term “halo” or “halogen” refers to fluorine (fluoro), chlorine (chloro), bromine (bromo) or iodine (iodo).

The present invention also relates to a use of a compound of Formula (I), or a pharmaceutically acceptable salt, tautomer, N-oxide, solvate, polymorph and/or prodrug thereof, in the manufacture of a medicament for causing weight loss in a subject:

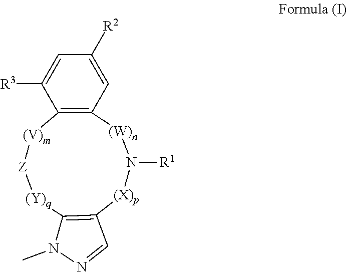

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

The present invention also relates to a compound of Formula (I), or a pharmaceutically acceptable salt, tautomer, N-oxide, solvate, polymorph and/or prodrug thereof, for use in a method for causing weight loss in a subject:

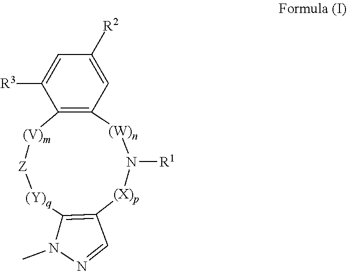

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

The term “causing weight loss” refers to a compound of Formula (I) leading to or resulting in a decrease in the weight of the subject.

The present invention also relates to a method for managing weight in a subject, the method including a step of administering to the subject a compound of Formula (I), or a pharmaceutically acceptable salt, tautomer, N-oxide, solvate, polymorph and/or prodrug thereof:

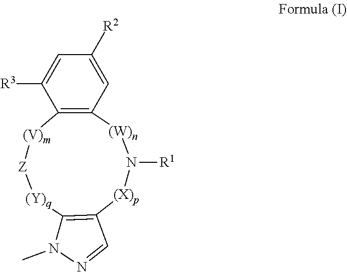

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

The present invention also relates to a use of a compound of Formula (I), or a pharmaceutically acceptable salt, tautomer, N-oxide, solvate, polymorph and/or prodrug thereof, in the manufacture of a medicament for managing weight in a subject:

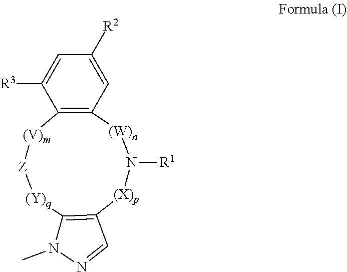

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

The present invention also relates to a compound of Formula (I), or a pharmaceutically acceptable salt, tautomer, N-oxide, solvate, polymorph and/or prodrug thereof, for use in a method for managing weight in a subject:

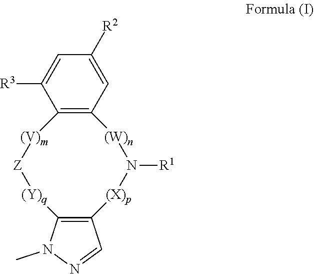

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

The term “managing weight” refers to a strategy for losing weight or maintaining a desired body weight over time. This strategy may lead to a decrease in the weight of the subject. The strategy may result in a stabilisation of the weight of the subject i.e. may prevent the patient from putting on additional weight.

The present invention also relates to a method for suppressing an appetite for food in a subject, the method including a step of administering to the subject a compound of Formula (I), or a pharmaceutically acceptable salt, tautomer, N-oxide, solvate, polymorph and/or prodrug thereof:

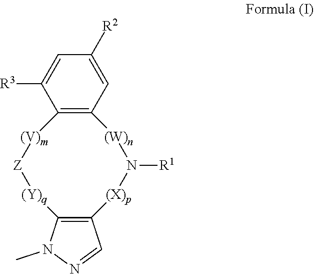

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

The present invention also relates to a use of a compound of Formula (I), or a pharmaceutically acceptable salt, tautomer, N-oxide, solvate, polymorph and/or prodrug thereof, in the manufacture of a medicament for suppressing an appetite for food in a subject:

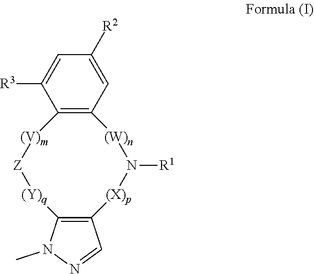

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

The present invention also relates to a compound of Formula (I), or a pharmaceutically acceptable salt, tautomer, N-oxide, solvate, polymorph and/or prodrug thereof, for use in a method for suppressing an appetite for food in a subject:

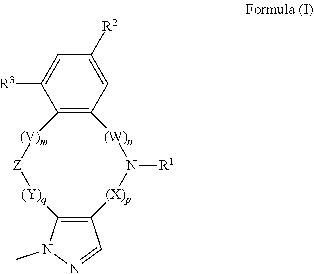

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

The term “suppressing an appetite for food” refers to reducing the desire to satisfy a need for food. This reduction in the desire to satisfy a need for food may lead to a decrease in the weight of the subject. The reduction in the desire to satisfy a need for food may result in a stabilisation of the weight of the subject i.e. may prevent the patient from putting on additional weight.

The present invention also relates to a method for reducing the consumption of food by a subject, the method including a step of administering to the subject a compound of Formula (I), or a pharmaceutically acceptable salt, tautomer, N-oxide, solvate, polymorph and/or prodrug thereof:

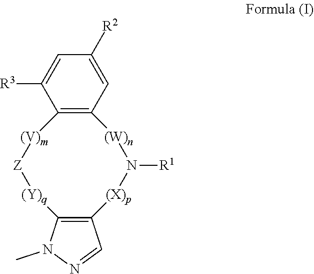

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

The present invention also relates to a use of a compound of Formula (I), or a pharmaceutically acceptable salt, tautomer, N-oxide, solvate, polymorph and/or prodrug thereof, in the manufacture of a medicament for reducing the consumption of food by a subject:

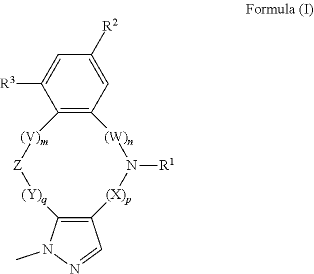

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

The present invention also relates to a compound of Formula (I), or a pharmaceutically acceptable salt, tautomer, N-oxide, solvate, polymorph and/or prodrug thereof, for use in a method for reducing, minimising or controlling the consumption of food by a subject:

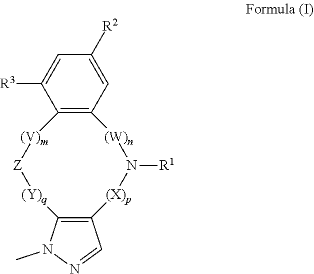

wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

The terms “reducing or minimising the consumption of food” refers to reducing the amount of food that is self-administered (i.e. eaten) by a subject as compared to the amount of food that is self-administered prior to the administration of a compound of the invention. This reduction in food consumption may also lead to a decrease in the weight of the subject. The reduction in food consumption may result in a stabilisation of the weight of the subject i.e. may prevent the patient from putting on additional weight.

The term “controlling” the consumption of food refers to controlling the amount of food that is self-administered by a subject, to a level that is compatible with the caloric requirements of the subject (and therefore prevents or reduces the likelihood that the subject will consume an amount of food that exceeds the caloric/energy requirements of the subject, leading to weight gain).

Without wishing to be bound by theory or mode of action, the present inventors believe that the compounds of formula (I) act at least partly by triggering satiety signals and reducing feeding in response to stomach distensions.

Disclosed herein are also aspects of the invention as described above, wherein the compound is of Formula (Ia):

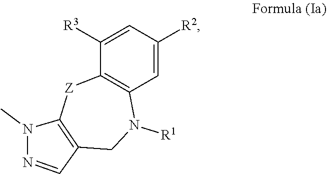

wherein:

Z is selected from: NH, O, S, S(O) or SO₂;

R¹ is selected from H or C(O)R⁴;
R² is selected from: H, a halogen, an optionally substituted C₁₋₅ alkyl, or an optionally substituted OC₁₋₅ alkyl;
R³ is selected from: H, a halogen, an optionally substituted C₁₋₅ alkyl, or an optionally substituted OC₁₋₅ alkyl; and
R⁴ is an optionally substituted C₁₋₅ alkyl, or a salt, tautomer, N-oxide, solvate, polymorph and/or prodrug thereof.

Disclosed herein are also aspects of the invention as described above, wherein the compound is of Formula (Ib):

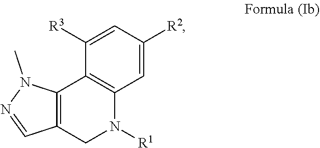

wherein:
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl, or a salt, tautomer, N-oxide, solvate, polymorph and/or prodrug thereof.

Disclosed herein are also aspects of the invention as described above, wherein the compound is of Formula (Ic):

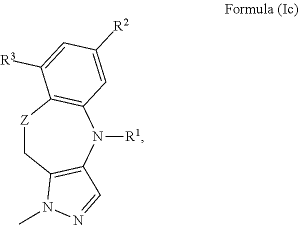

wherein:
Z is selected from: NH, O, S, S(O) or SO₂;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

Disclosed herein are also aspects of the invention as described above, wherein the compound is of Formula (Id):

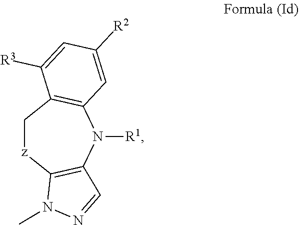

wherein:
Z is selected from: NH, O, S, S(O) or SO₂;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

Disclosed herein are also aspects of the invention as described above, wherein the compound is of Formula (Ie):

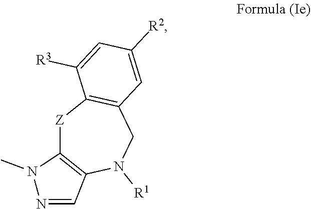

wherein:
Z is selected from: NH, O, S, S(O) or SO₂;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

In one embodiment, Z is NH.

In another embodiment, Z is O.

In another embodiment, Z is S.

In another embodiment, Z is S(O).

In another embodiment, Z is SO₂.

In one embodiment, R¹ is hydrogen.

In another embodiment, R¹ is C(O)R⁴.

In another embodiment, R¹ is C(O)R⁴ and R⁴ is an optionally substituted C₁₋₅ alkyl selected from any one of: methyl, ethyl, propyl, isopropyl, n-butyl, sec-butyl, tert-butyl, n-pentyl, neopentyl, iso-pentyl and tert-pentyl.

In another embodiment, R¹ is C(O)R⁴ and R⁴ is an optionally substituted methyl.

In one embodiment, R² is hydrogen.

In another embodiment, R² is a hydroxyl group.

In another embodiment, R² is a halogen.

In another embodiment, R² is fluorine.

In another embodiment, R² is chlorine.

In another embodiment, R² is an optionally substituted C₁₋₅ alkyl.

In another embodiment, R² is an optionally substituted methyl.

In another embodiment, R² is an optionally substituted OC₁₋₅ alkyl.

In another embodiment, R² is an optionally substituted methoxy group.

In one embodiment, R³ is hydrogen.

In another embodiment, R³ is a hydroxyl group.

In another embodiment, R³ is a halogen.

In another embodiment, R³ is fluorine.

In another embodiment, R³ is chlorine.

In another embodiment, R³ is an optionally substituted C₁₋₅ alkyl.

In another embodiment, R³ is an optionally substituted methyl.

In another embodiment, R³ is an optionally substituted OC₁₋₅ alkyl.

In another embodiment, R³ is an optionally substituted methoxy group.

In some embodiments, R¹ and R³ are H, R² is H or halo and Z is NH.

In some embodiments, R¹, R² and R³ are H and Z is NH.

In some embodiments, V, Y and W are direct bonds and X is CH₂, and m, n, p and q are each 1.

In some embodiments, R¹ and R³ are H, R² is H or halo, Z is NH, V, Y and W are direct bonds and X is CH₂, and m, n, p and q are each 1.

In some embodiments, X is CH₂, p is 1, and q, m and n are each 0.

In one embodiment the compound of Formula (I) is selected from:

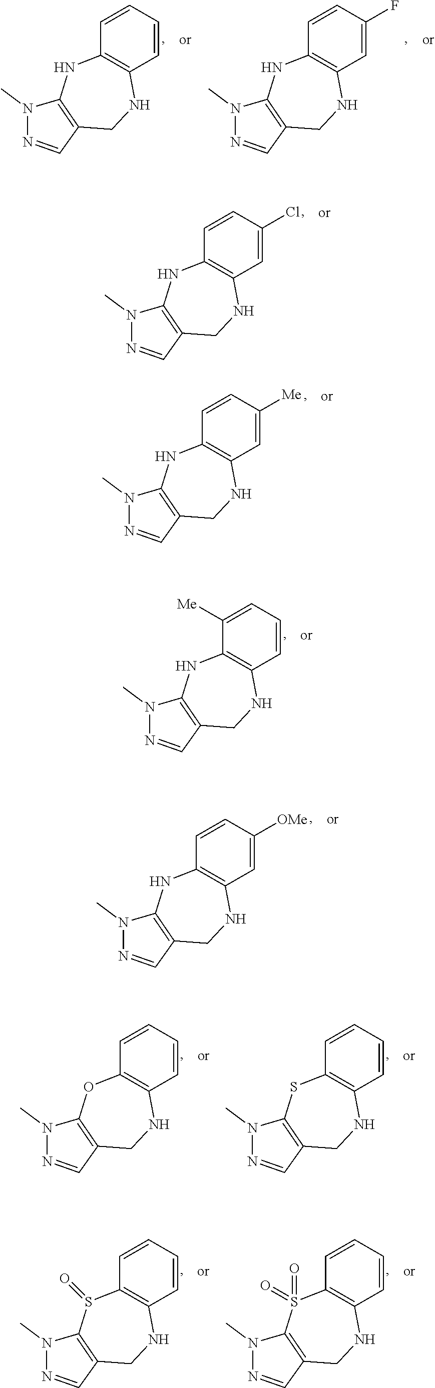

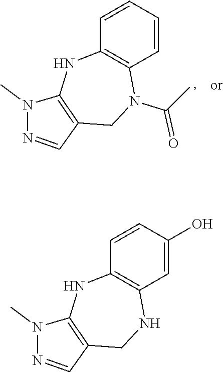

or a salt, tautomer, N-oxide, solvate, polymorph and/or prodrug thereof.

In another embodiment compound of Formula (I) is selected from:

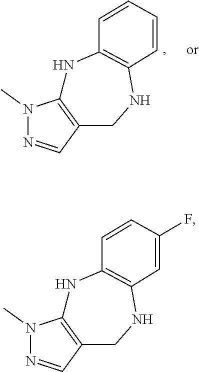 [SMILES: Cn1ncc2c1Nc1ccccc1NC2 ; Cn1ncc2c1Nc1ccc(F)cc1NC2]

or a pharmaceutically acceptable salt, tautomer, N-oxide, solvate, polymorph and/or prodrug thereof. For example, in one embodiment the compound of Formula (I) is:

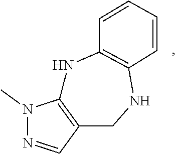

or a pharmaceutically acceptable salt, tautomer, N-oxide, solvate, polymorph and/or prodrug thereof. In another embodiment, the compound of Formula (I) is:

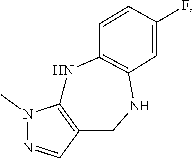 [SMILES: Cn1ncc2c1Nc1ccc(F)cc1NC2]

or a pharmaceutically acceptable salt, tautomer, N-oxide, solvate, polymorph and/or prodrug thereof.

### Pharmaceutically Acceptable Compounds

The compounds of Formula (I) or salts or prodrugs thereof used in the treatments described herein are pharmaceutically acceptable. Herein, “pharmaceutically acceptable” means that compounds of Formula (I), or salts or prodrugs thereof, along with carriers, diluents or excipients, must be compatible with the other ingredients of a formulation, and typically not deleterious to the recipient. The compounds of Formula (I), or pharmaceutically acceptable salts or prodrugs thereof, should be able to contact tissues of a recipient without excessive toxicity, irritation, allergic response or other potential complications commensurate with a reasonable benefit/risk ratio identified by a skilled medical professional or veterinarian. In addition a compound of Formula (I), or a pharmaceutically acceptable salt, tautomer, N-oxide, solvate, polymorph and/or prodrug thereof, should be compatible with a carrier and/or excipient in any composition that is to be delivered as a medicament to an individual, for example to an animal or human being.

Suitable pharmaceutically acceptable salts of Formula (I) include, but are not limited to: salts of pharmaceutically acceptable inorganic acids such as: hydrochloric (including monohydrochloride, dihydrochloride, trihydrochloride etc), sulfuric, phosphoric, nitric, carbonic, boric, sulfamic, and hydrobromic acids; or salts of pharmaceutically acceptable organic acids such as acetic, propionic, butyric, tartaric, maleic, hydroxymaleic, fumaric, isethionic, malic, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids.

Base salts include, but are not limited to: those formed with pharmaceutically acceptable cations, such as: sodium, potassium, lithium, calcium, magnesium, zinc, ammonium and alkylammonium; salts formed from triethylamine; alkoxyammonium salts such as those formed with ethanolamine; and salts formed from ethylenediamine, choline or amino acids such as arginine, lysine or histidine.

General information on types of pharmaceutically acceptable salts and their formation is known to those skilled in the art and is as described in general texts such as “Handbook of Pharmaceutical salts” P. H. Stahl, C. G. Wermuth, 1^{st} edition, 2002, Wiley-VCH.

Basic nitrogen-containing groups in Formula (I) (or basic nitrogen-containing groups in a compound of Formula (Ia), Formula (Ib), Formula (Ic), Formula (Id) or Formula (Ie)), may be quarternised with such agents as lower alkyl halide, such as methyl, ethyl, propyl, and butyl chlorides, bromides and iodides; dialkyl sulfates like dimethyl and diethyl sulfate; and others known in the art.

In one embodiment the compound of Formula (I) is a pharmaceutically acceptable salt of a compound selected from:

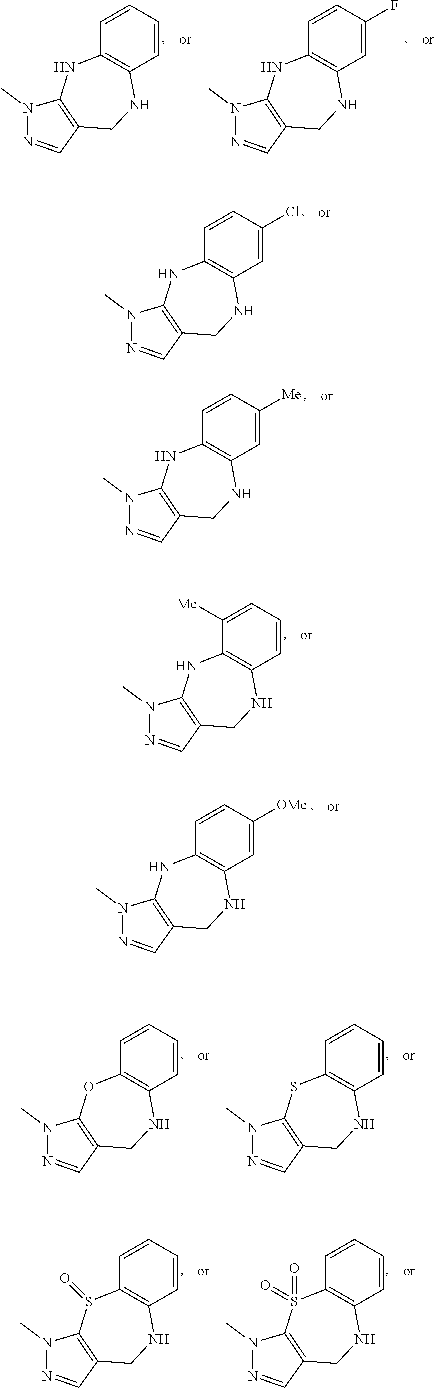

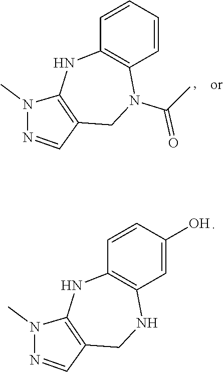

In another embodiment, the compound of Formula (I) is a pharmaceutically acceptable salt of a compound selected from:

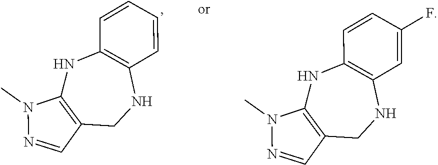 [SMILES: Cn1ncc2c1Nc1ccccc1NC2 ; Cn1ncc2c1Nc1ccc(F)cc1NC2]

For example, in one embodiment, the salt of a pharmaceutically acceptable compound of Formula (I) is a salt of

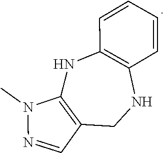

Whilst, in yet another embodiment, the pharmaceutically acceptable salt of a compound of Formula (I) is a salt of

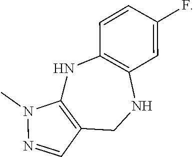 [SMILES: Cn1ncc2c1Nc1ccc(F)cc1NC2]

In one embodiment the compound of Formula (I) is a pharmaceutically acceptable hydrochloride salt. The hydrochloride salt may be a dihydrochloride salt of the compound of Formula (I).

In one embodiment the compound of Formula (I) is a compound of Formula (Ia) in the form of a hydrochloride salt.

In one embodiment the compound of Formula (I) is a compound of Formula (Ib) in the form of a hydrochloride salt.

In one embodiment the compound of Formula (I) is a compound of Formula (Ic) in the form of a hydrochloride salt.

In one embodiment the compound of Formula (I) is a compound of Formula (Id) in the form of a hydrochloride salt.

In one embodiment the compound of Formula (I) is a compound of Formula (Ie) in the form of a hydrochloride salt.

In one embodiment the compound of Formula (I) is a pharmaceutically acceptable hydrochloride salt of a compound selected from:

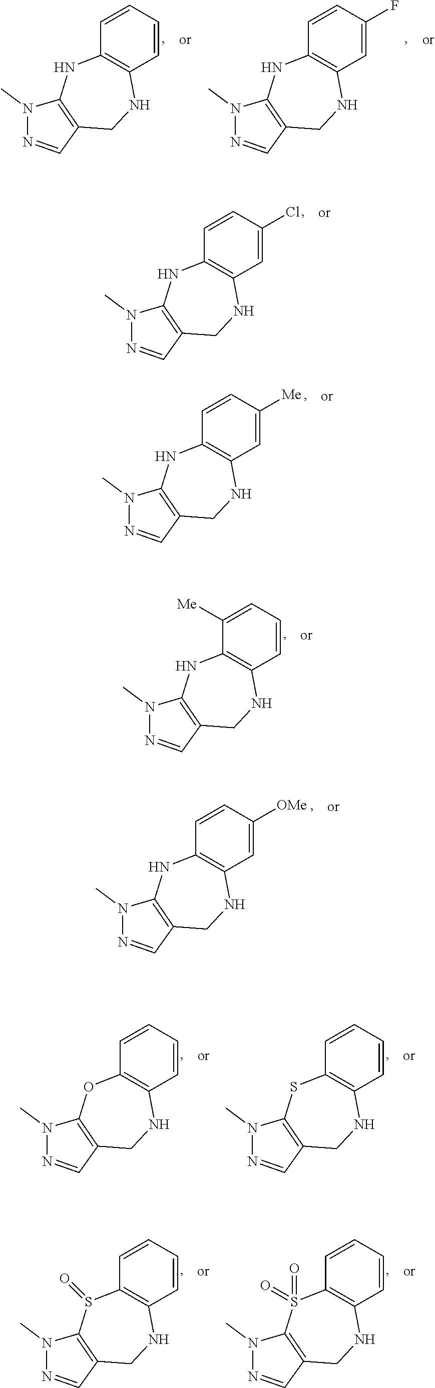

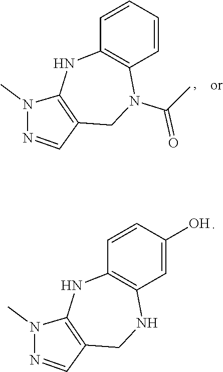

In another embodiment, the compound of Formula (I) is a pharmaceutically acceptable hydrochloride salt of a compound selected from:

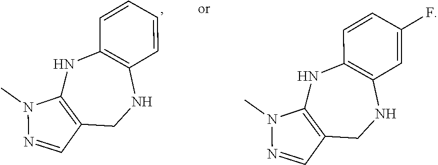 [SMILES: Cn1ncc2c1Nc1ccccc1NC2 ; Cn1ncc2c1Nc1ccc(F)cc1NC2]

For example, in one embodiment, the salt of a compound of Formula (I) is a pharmaceutically acceptable hydrochloride salt of

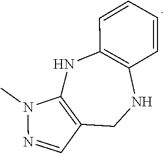

Whilst, in yet another embodiment, the salt of a compound of Formula (I) is a pharmaceutically acceptable hydrochloride salt of

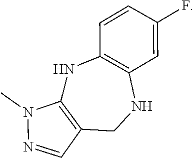 [SMILES: Cn1ncc2c1Nc1ccc(F)cc1NC2]

In one embodiment the compound of Formula (I) is a pharmaceutically acceptable prodrug.

In one embodiment the compound of Formula (Ia) is a pharmaceutically acceptable prodrug.

In one embodiment the compound of Formula (Ib) is a pharmaceutically acceptable prodrug.

In one embodiment the compound of Formula (Ic) is a pharmaceutically acceptable prodrug.

In one embodiment the compound of Formula (Id) is a pharmaceutically acceptable prodrug.

In one embodiment the compound of Formula (Ie) is a pharmaceutically acceptable prodrug.

Prodrugs include compounds wherein an amino acid residue, or a polypeptide chain of two or more (e.g., two, three or four) amino acid residues, is covalently joined to free amino, hydroxy or carboxylic acid groups of compounds of Formula (I). The amino acid residues include the 20 naturally occurring amino acids commonly designated by three letter symbols and also include: 4-hydroxyproline, hydroxylysine, demosine, isodemosine, 3-methylhistidine, norvaline, beta-alanine, gamma-am inobutyric acid, citrulline, homocysteine, homoserine, ornithine and methionine sulfone. Prodrugs also include compounds wherein carbonates, carbamates, amides and alkyl esters, which may be covalently bonded to the above substituents of Formula (I) through the carbonyl carbon prodrug side chain. Prodrugs also include phosphate derivatives of compounds of Formula (I) (such as acids, salts of acids, or esters) joined through a phosphorus-oxygen bond to a free hydroxyl of compounds of Formula (I).

Any nitrogen atom in a compound of Formula (I) may exist as an N-oxide.

The compounds of Formula (I) or salts, tautomers, N-oxides, polymorphs or prodrugs thereof may be provided in the form of solvates. Solvates contain either stoichiometric or non-stoichiometric amounts of a solvent, and may be formed during the process of crystallization with pharmaceutically acceptable solvents such as water, alcohols such as methanol, ethanol or isopropyl alcohol, DMSO, acetonitrile, dimethyl formamide (DMF) and the like with the solvate forming part of the crystal lattice by either non-covalent binding or by occupying a hole in the crystal lattice. Hydrates are formed when the solvent is water, alcoholates are formed when the solvent is alcohol. Solvates of the compounds of the present invention can be conveniently prepared or formed during the processes described herein. In general, the solvated forms are considered equivalent to the unsolvated forms for the purposes of the compounds and methods provided herein.

The compound of Formula (I) or salts, tautomers, N-oxides, solvates and/or prodrugs thereof that form crystalline solids may demonstrate polymorphism. All polymorphic forms of the compounds, salts, tautomers, N-oxides, solvates and/or prodrugs may be used in the methods of the invention.

The compound of Formula (I) may demonstrate tautomerism. Tautomers are two interchangeable forms of a molecule that typically exist within an equilibrium. Any tautomers of the compounds of Formula (I) may be used in the methods of the invention.

Methods for making compounds of Formula (I) are described in PCT/AU2016/050588.

### Administration

The compounds of Formula (I), or a pharmaceutically acceptable salt, tautomer, N-oxide, solvate, polymorph and/or prodrug thereof, as defined herein, may be administered by any suitable means, for example, orally, rectally, nasally, vaginally, topically (including buccal and sub-lingual), parenterally, such as by subcutaneous, intraperitoneal, intravenous, intramuscular, or intracisternal injection, inhalation, insufflation, infusion or implantation techniques (e.g., as sterile injectable aqueous or non-aqueous solutions or suspensions).

The compounds of the invention may be provided as pharmaceutical compositions including those for oral, rectal, nasal, topical (including buccal and sub-lingual), parenteral administration (including intramuscular, intraperitoneal, subcutaneous and intravenous), or in a form suitable for administration by inhalation or insufflation. The compounds of Formula (I), or a pharmaceutically acceptable salt, tautomer, N-oxide, solvate, polymorph and/or prodrug thereof, together with a conventional adjuvant, carrier or diluent, may thus be placed into the form of pharmaceutical compositions and unit dosages thereof, and in such form may be employed as solids, such as tablets or filled capsules, or liquids as solutions, suspensions, emulsions, elixirs or capsules filled with the same, all for oral use, or in the form of sterile injectable solutions for parenteral (including subcutaneous) use.

The pharmaceutical compositions for the administration of the compounds of this invention may conveniently be presented in dosage unit form and may be prepared by any of the methods well known in the art of pharmacy (See, for example, Remington: The Science and Practice of Pharmacy, 21st Ed., 2005, Lippincott Williams & Wilkins). All methods include the step of bringing the active ingredient, for example a compound defined by Formula (I), or a pharmaceutically acceptable salt, tautomer, N-oxide, solvate, polymorph and/or prodrug thereof, into association with the carrier which constitutes one or more accessory ingredients. In general, the pharmaceutical compositions are prepared by uniformly and intimately bringing the active ingredient, for example a compound defined by Formula (I), or a pharmaceutically acceptable salt, tautomer, N-oxide, solvate, polymorph and/or prodrug thereof, into association with a liquid carrier or a finely divided solid carrier or both, and then, if necessary, shaping the product into the desired formulation. In the pharmaceutical composition the active object compound is included in an amount sufficient to produce the desired effect.

Compounds of Formula (I), or a pharmaceutically acceptable salt, tautomer, N-oxide, solvate, polymorph and/or prodrug thereof, may be administered in doses of about 0.001 mg/kg, about 0.005 mg/kg, about 0.01 mg/kg, about 0.05 mg/kg, about 0.1 mg/kg, about 0.15 mg/kg, about 0.2 mg/kg, about 0.5 mg/kg, about 1 mg/kg, about 2 mg/kg, about 3 mg/kg, about 5 mg/kg, about 10 mg/kg, about 15 mg/kg, about 20 mg/kg, about 25 mg/kg or about 30 mg/kg or more, of the body weight of the subject. In some embodiments, the dose may be from any of these amounts to any other amount, such as from about 0.001 mg/kg to about 30 mg/kg, about 0.2 mg/kg to about 30 mg/kg or about 0.2 mg/kg to about 10 mg/kg. It will be understood, however, that the specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Compounds of Formula (I), or a pharmaceutically acceptable salt, tautomer, N-oxide, solvate, polymorph and/or prodrug thereof, may be provided in an “effective amount”, for example when an appropriate compound is added to a pharmaceutical composition. “Effective amount” is taken to mean an amount of a compound that will elicit a desired biological or medical response of a tissue, system, animal or human that is being sought by the researcher, veterinarian, medical doctor or other clinician administering the compound of a composition including the compound. In some embodiments, the effective amount may be a “therapeutically effective amount” wherein the amount of the object active compound is effective to treat the condition and/or symptom thereof that has manifested in the subject. In other embodiments, the effective amount may be a “prophylactically effective amount” wherein the amount of the object active compound is sufficient to prophylactically treat and/or prevent the onset of the condition and/or a symptom thereof or, if a symptom emerges, cause the severity of the condition and/or symptom thereof to be at a reduced level compared to the average severity of the condition and/or symptom thereof in a population of subjects not having received treatment with the compound of Formula (I) and/or a pharmaceutically acceptable salt, tautomer, N-oxide, solvate, polymorph and/or prodrug thereof.

An “effective amount” is that amount of a compound of Formula (I), or a pharmaceutically acceptable salt, tautomer, N-oxide, solvate, polymorph and/or prodrug thereof, provided herein, the administration of which to a subject, either in a single dose or as part of a series, is effective for weight management. An amount is effective, for example, when its administration results in one or more of weight loss, management of weight, suppression of appetite for food, control of food intake, loss of body fat, prevention or modulation of hypoglycemia, prevention or modulation of hyperglycemia, promotion of insulin synthesis, or reduction in food intake.

The “effective amount” will be dependent on a number of factors, including the efficacy of the particular compound, physical condition of the subject to be treated, the extent of weight loss or weight maintenance desired, the formulation of the compound, and/or a professional assessment of the medical situation. The subject's weight and age may also be a factor for the person skilled in the art when determining the amount of compound that the subject should receive.

The phrases “administration of” and or “administering a” compound should be understood to mean providing a compound of Formula (I) (or a compound of Formula (Ia), Formula (Ib), Formula (Ic), Formula (Id) or Formula (Ie)), or a pharmaceutically acceptable salt, tautomer, N-oxide, solvate, polymorph and/or prodrug thereof, to a subject in need thereof.

As provided herein, beneficial or desired clinical results from the disclosed compound of Formula (I), or a pharmaceutically acceptable salt, tautomer, N-oxide, solvate, polymorph and/or prodrug thereof, include, without limitation, reduced body weight, decreased weight-gain, reduced appetite, weight loss, management of weight, suppression of an appetite for food, controlled food intake, reduced or stabilized serum glucose and serum insulin levels, amelioration, palliation, stabilization, diminishment of extent of obesity-related diseases, or a delay or slowing of obesity related disease progression. Either therapeutic or preventative measures may be achieved. Those in need of treatment include those already with the disorder as well as those in which the disorder is to be prevented. By treatment is meant inhibiting or reducing an increase in obesity-related symptoms (e.g. weight gain) when compared to the absence of treatment, and is not necessarily meant to imply complete cessation of the relevant condition.

### Subjects

The subjects requiring treatment in accordance with the methods of the present invention are those individuals who, as a result of excessive food consumption, are at risk of gaining weight to become overweight; those who are already overweight; if already overweight, those at risk of becoming obese; as well as those who are already obese (including morbidly obese). Furthermore, the subject requiring treatment in accordance with the present invention may be an individual who is not currently overweight, but who is at risk of becoming overweight. The subject may already be overweight. The subject may be obese.

In one embodiment the subject is obese. In one embodiment, the subject is morbidly obese.

In one embodiment the subject is overweight.

In one embodiment the subject is at risk of becoming overweight.

The terms “overweight” and “obesity” (or “obese”) are broadly defined as “abnormal or excessive fat accumulation that presents a risk to health” (World Health Organization). There is not a single index or measure that is applied to determine whether a patient is “overweight” or “obese”, as the amount of fat accumulation that presents a risk to health varies for humans and for non-humans. For instance, for humans, the amount of fat accumulation that presents a risk to health varies with age and ethnicity. However, a person skilled in the art would be able to assess if a subject is considered to be overweight or obese using their own skill and knowledge, and the appropriate guide or index for the patient in question.

It will be understood that weight gain, excessive weight and obesity may arise from a caloric intake that exceeds the energy expenditure of the individual. Weight gain, excessive weight and obesity may also arise from the type of food consumed in excess by a subject.

The subject requiring treatment in accordance with the present invention may be a human adult, human child or human juvenile (including teenagers). Alternatively, the subject may be a companion animal, such as a cat, dog, mouse or other.

Obesity is a medical condition in which excess body fat has accumulated to the extent that it may have an adverse effect on health. It is defined by the body mass index (BMI) and further evaluated in terms of fat distribution via the waist-hip ratio and total cardiovascular risk factors. BMI is closely related to both percentage body fat and total body fat. In children, a healthy weight varies with age and sex.

Body mass index (BMI) is a simple index of weight-for-height that is commonly used for human adults to assess whether a patient is “overweight” or “obese”. It is defined as an individual's weight in kilograms divided by the square of his or her height in meters (kg/m²). The most commonly used definitions, established by the World Health Organization (WHO) in 1997 and published in 2000, provide the following classifications based on BMI: overweight=human adult with BMI of between about 25 to about 29.99 kg/m²; class I obesity=a human adult with BMI from between about 30 kg/m² to about 34.99 kg/m²; severe obesity (class II obesity)=a human adult with BMI of between about 35 kg/m² to about 39.99 kg/m²; morbid obesity (class III obesity)=a human adult with BMI of between about 40 kg/m² and 44.99 kg/m². Some physicians/clinicians/surgeons may also refer to super obesity, being a BMI of greater than about 45 kg/m² in a human adult subject. Further, some surgeons may refer to morbid obesity as being a BMI of 35 kg/m² and experiencing obesity-related health conditions or ≥40-44.9 kg/m². A human adult subject whose BMI is increasing but is currently not greater than or equal to 25 is considered to be “at risk of becoming overweight”.

As Asian populations develop negative health consequences at a lower BMI than Caucasians, some nations have redefined obesity; Japan has defined obesity as any BMI greater than 25 kg/m² while China uses a BMI of greater than 28 kg/m².

The BMI as an overweight and obesity indicator is also used differently for children. For children aged between 5 to 19 years of age, a subject whose BMI is greater than 1 standard deviation above the WHO Growth Reference median for the age group of the subject is considered to be overweight. For children aged between 5 to 19 years of age, a subject whose BMI is greater than 2 standard deviations above the WHO Growth Reference median for the age group of the subject is considered to be obese. For children under 5 years of age, a subject whose BMI is greater than 2 standard deviations above the WHO Growth Reference median for the age group of the subject is considered to be overweight. For children under 5 years of age, a subject whose BMI is greater than 3 standard deviations above the WHO Growth Reference median for the age group of the subject is considered to be obese.

As for humans, there is not a single index or measure for the assessment of excess weight and obesity in animals such as dogs and cats. According to one commonly-used index, a dog or cat is considered to be overweight when its weight is greater than 15% above ideal weight, and a dog or cat is considered to be obese when its weight is greater than 30% above ideal weight.

Obesity increases the likelihood of various diseases and conditions, particularly cardiovascular diseases, type 2 diabetes, obstructive sleep apnea, certain types of cancer, osteoarthritis, and depression. Besides using BMI as an indicator of excessive weight, and obesity, a number of biomarkers can be measured to assess the effect of weight management regime. Common biomarkers include glucose and insulin in blood plasma, triglycerides, total cholesterol, non-esterified fatty acids (NEFA), and beta-hydroxybutyrate.

Other indicators of excessive weight and obesity include waist circumference and percentage lean body mass. The skilled person will be familiar with methods for assessing these indicators and measures of these indicators which are typically indicative of being overweight or obese.

The subject may be at risk of increasing in weight (i.e. becoming overweight or obese) due to an uncontrolled motivation to consume food. This motivation may arise from the fact that some foods, such as highly palatable foods (i.e. those that are high in sugar and/or high in fat), are particularly attractive from an organoleptic perspective in their own right. The subject may have any disorder that causes excessive food consumption (i.e., hyperalimentation). The desire to consume excessive amounts of food may arise as a result of a disorder that leads to weight gain through excessive consumption of food. Accordingly, the subject may have a disorder that leads to weight gain through excessive consumption of food, such as a disorder selected from compulsive overeating disorder, binge eating disorder (BED), subclinical binge eating, anxiety, depression, Prader-Willi syndrome and metabolic syndrome (also known as syndrome X or insulin-resistance syndrome). The subject may have other obesity-related metabolic disorders. Examples of other obesity-related disorders include without limitation: insulin resistance, glucose intolerance, pre-diabetes, increased fasting glucose, type 2 diabetes, hypertension, dyslipidemia (or a combination of these metabolic risk factors), glucagonomas, cardiovascular diseases such as congestive heart failure, atherosclerosis, arteriosclerosis, coronary heart disease, or peripheral artery disease, stroke, respiratory dysfunction, or renal disease.

As used herein, the term “food” will be understood to refer to any solid or liquid substance consumed by the subject, wherein the substance is a source of calories. It will therefore be understood that the term “food” includes beverage as well as non-beverage foodstuffs. The food may be a highly palatable food (i.e. a high-fat food and/or a high-sugar food). Examples of these foods include margarine, butter, pizza, cakes, biscuits, chocolate, French fries, potato chips, low-fat yoghurt, ice cream, burgers, confectionery (“lollies”), pastries, and high caloric drinks (such as fruit juices and other fruit drinks, soft drinks, energy drinks, sweetened cordials, sweetened waters).

In any method or use described herein, the food for which the appetite is suppressed, or for which intake is controlled, reduced or minimised, is preferably selected from the group consisting of: a high-fat food, a high-sugar food or a food that is high in both fat and sugar. In further embodiments, the high-sugar food may be a high-caloric drink (including a soft drink, fruit juice, fruit purée, formulated beverage or the like). The high-caloric drink may be alcoholic or non-alcoholic. A formulated beverage will typically be understood to include a non-carbonated, ready-to-drink, flavoured beverage that: is water-based; and contains added vitamins or minerals or both vitamins and minerals; and may also contain fruit juice; fruit purée; concentrated fruit juice; concentrated fruit purée; comminuted fruit; orange peel extract; and sugars.

The recipients of a pharmaceutically acceptable compound of Formula (I), or a pharmaceutically acceptable salt, tautomer, N-oxide, solvate, polymorph and/or prodrug thereof; or a pharmaceutical composition including a compound of Formula (I) or a pharmaceutically acceptable salt, tautomer, N-oxide, solvate, polymorph and/or prodrug thereof, can be human beings, male or female.

Alternatively the recipients of: a compound of Formula (I), or a pharmaceutically acceptable salt, tautomer, N-oxide, solvate, polymorph and/or prodrug thereof; or a pharmaceutical composition including a compound of Formula (I), or a pharmaceutically acceptable salt, tautomer, N-oxide, solvate, polymorph and/or prodrug thereof, can also be a non-human animal. “Non-human animals” or “non-human animal” is directed to the kingdom Animalia, excluding humans, and includes both vertebrates and invertebrates, male or female, and includes: warm blooded animals, including mammals (including but not limited to primates, dogs, cats, cattle, pigs, sheep, goats, rats, guinea pigs, horses, or other bovine, ovine, equine, canine, feline, rodent or murine species), birds, insects, reptiles, fish and amphibians.

The recipients of the compounds and compositions are referred herein with the interchangeable terms “patient”, “recipient”, “individual”, and “subject”. These four terms are used interchangeably and refer to any human or animal (unless indicated otherwise), as defined herein.

### EXAMPLES

A hydrochloride salt of the compound of the following formula:

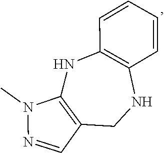

was utilised in the following examples and was given the code SOC-1.

### Progressive Ratio Consumption of a High-Sugar High-Fat Food by Non-Human Primates

Individuals who are overweight or obese frequently report intense cravings for, and motivation to, consume food. Here we model the motivational aspects that can drive excessive food-seeking and consumption, which can lead to overweight and obesity by studying rhesus monkeys who must respond under a progressive ratio to receive M&Ms (which are high in both fat and sugar). A progressive ratio requires subjects to work increasingly harder to obtain reinforcement, modelling the intense motivation and craving characteristic of excessive food-seeking and consumption in individuals who are obese, overweight or at risk of being overweight [1, 2].

Five female rhesus monkeys were trained to press a lever to self-administer M&Ms. Under a progressive ratio, subjects responded for a reinforcer of either 1 or 5 M&Ms. The ratio began at either 25 (two subjects) or 50 (three subjects) responses per reinforcement and increased by 40% after each reinforcer delivery. SOC-1 was administered IM using a within-subjects design whereby all monkeys received doses of 0, 1, 2 and 3 mg/kg SOC-1. One dose per subject was administered prior to each progressive ratio test session. Breakpoint refers to the number of times the monkeys would press the lever to receive a single reinforcer before giving up.

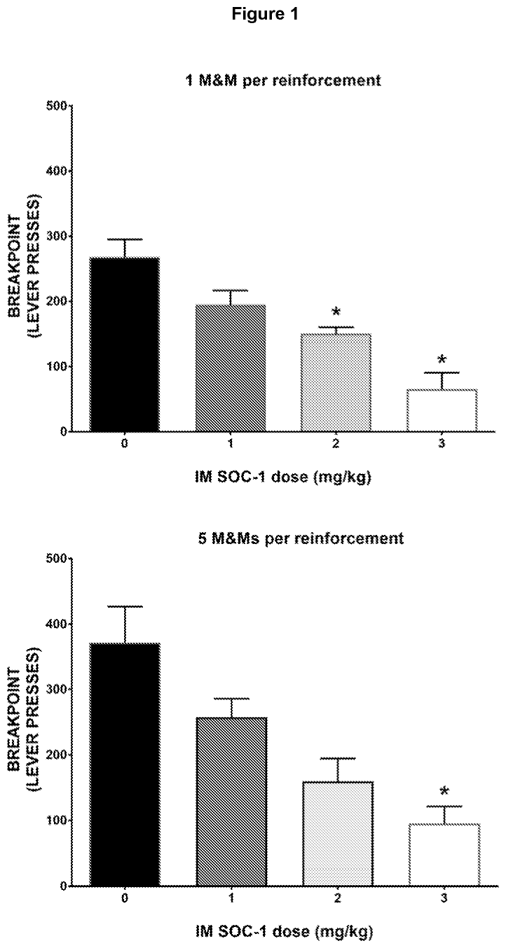

Data from this experiment are presented in FIG. 1. When each reinforcement was 1 M&M, SOC-1 significantly reduced the breakpoint at doses of 2 (p=0.015) and 3 (p=0.014) mg/kg and trend analysis revealed there was a significant linear decrease in breakpoint with increasing doses of SOC-1 (p=0.012). When each reinforcement was 5 M&Ms, SOC-1 significantly reduced the breakpoint at 3 mg/kg (p=0.021) and trend analysis revealed there was a significant linear decrease in breakpoint with increasing doses of SOC-1 (p=0.025).

### Sucrose Self-Administration

This model induces high levels of sucrose consumption and sucrose seeking in rats, akin to the unhealthy high sugar diets that are a major determinant of obesity. Excessive consumption of sugar-sweetened beverages are especially associated with increased risk of obesity and type 2 diabetes [3]. Thus, the model chosen here is particularly well suited as it involves escalating voluntary consumption of high-sucrose solution by rats, providing both face and construct validity.

Male Sprague Dawley rats were water deprived for 19.5 hours before the start of sucrose self-administration. Water deprivation continued for the first five days of self-administration. Initially, rats were trained during daily 30 minute FR-1 scheduled sessions to lever press for sucrose (10%). Stable responding under the FR-1 schedule was achieved within 10 days. Rats were then transferred to an FR2 and FR3 schedule for a minimum of three days, and then an FR5 schedule until lever pressing stabilised. Subsequently SOC-1 testing began. Each test day was separated by 2-3 FR-5 scheduled sessions. Rats received SOC-1 in an ascending dose sequence (0, 5, and 10 mg/kg, ip), which was administered 15 minutes prior to the test session. On the final test session, rats received a vehicle injection to ensure rats continued to self-administer sucrose at high levels when not receiving treatment with SOC-1.

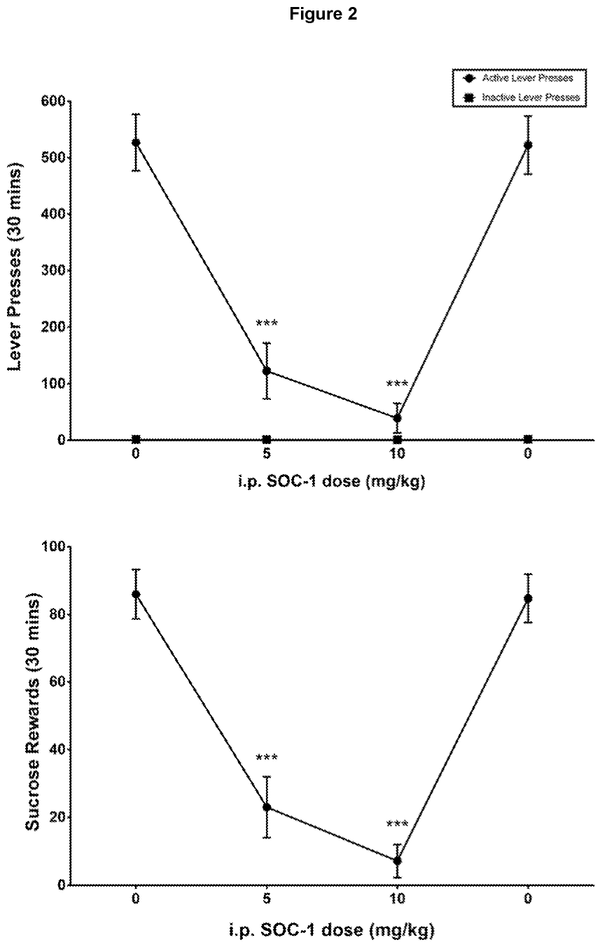

Data from this experiment are presented in FIG. 2. SOC-1 significantly decreased active lever presses and number of sucrose rewards received at doses of both 5 and 10 mg/kg (all p<0.001). SOC-1 had no significant effect on inactive lever presses.

### Reinstatement of Sucrose-Seeking

Sugar is a highly palatable food component. This contributes greatly to the maintenance of the high sugar diets that can lead to obesity. Once a healthier diet has been established, including cessation of consumption of high sugar foods, it can be difficult for individuals to maintain that healthier diet. Specifically, after a period of not consuming the high sugar foods individuals can again start seeking out and consuming those foods. Here we model this resumption of high-sugar diet consumption using a rat model of reinstatement of sucrose self-administration to assess whether SOC-1 might reduce motivation to resume consumption of high-sugar foods.

Male Sprague Dawley rats were water deprived for 19.5 hours before the start of sucrose self-administration. Water deprivation continued for the first five days of self-administration. Rats were allowed to acquire self-administration of sucrose (10%) during 30-minute FR-1 scheduled sessions for 20 days. Following this, rats were exposed to daily 30-minute extinction sessions. Rats met the extinction criteria (minimum of 10 days and less than 15 active lever presses per session for 2 consecutive days) after 10 sessions. Once the extinction criteria were met, rats underwent reinstatement testing. Each reinstatement test session was separated by two extinction days. Prior to each reinstatement session, rats received an injection of vehicle or SOC-1. Fifteen minutes later, they were placed in the testing chamber, where they had access to 0.3 ml sucrose, which they could lever press for. Rats received SOC-1 in an ascending dose sequence (0, 5, 10 mg/kg, ip). On the reinstatement session subsequent to SOC-1 dose testing, an additional vehicle session was conducted to ensure that rats continued to reinstate to sucrose-seeking behaviour when no treatment was given.

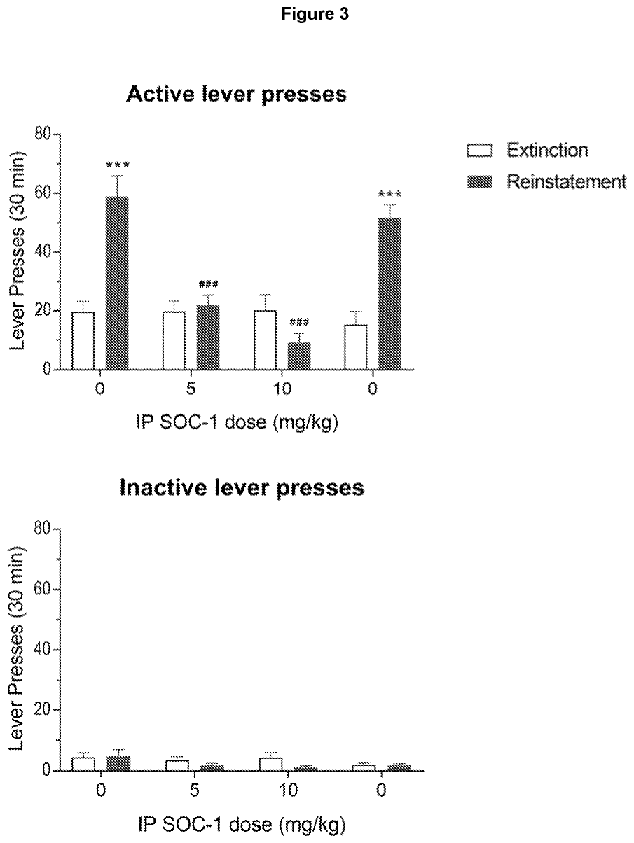

Data from this experiment are presented in FIG. 3. When vehicle (0 mg/kg) SOC-1 was administered rats showed significant reinstatement of sucrose-seeking (p<0.001). SOC-1 completely prevented reinstatement of sucrose seeking at doses of 5 and 10 mg/kg (vs 0 mg/kg reinstatement all p<0.001). Reinstatement had no significant effect on inactive lever presses nor did any dose of SOC-1.

### Extinction of Food Reward-Associated Lever Pressing in Food Restricted Rats

Adult male Long-Evans rats were allowed to feed freely until they reached a target weight of 320-340 g. Rats were then maintained at this weight by restricting feeding. Once target weight had been reached, each rat was placed in an operant test chamber and was trained to press the right lever to receive a 45 mg food pellet under an FR1 schedule of reinforcement during daily 15 min sessions.

Once stable responding (right-side lever pressing+/−10% of the mean of the previous three sessions) had been established, drug testing commenced. Rats were pre-treated with SOC-1 (0, 5, 10, 20 mg/kg) administered by oral gavage (p.o.) 30 min prior to the start of the session. During the test session lever pressing did not result in food pellet delivery.

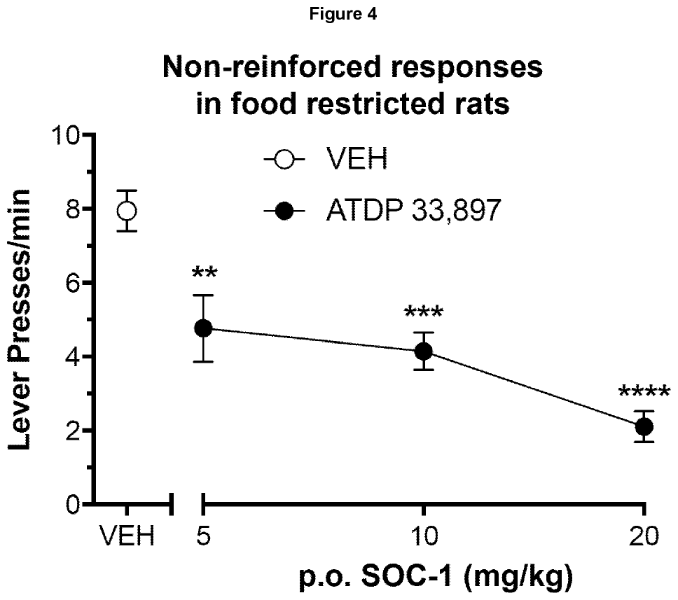

Data from this experiment are presented in FIG. 4. All doses tested significantly reduced non-reinforced responding for a food pellet in food restricted rats. These findings indicate SOC-1 can facilitate extinction of food-seeking behaviour in rats with heightened motivation to seek food.

### Inhibition of Weight Gain in Rats

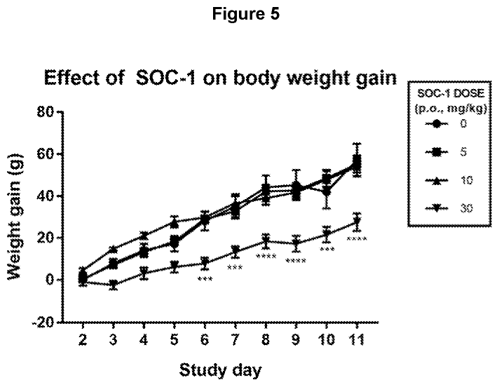

20 adult male Sprague Dawley rats were given ad libitum access to standard rat chow in their home cage over the course of the experiment. Rats were divided into four equally sized groups and were treated once daily with either 0, 5, 10 or 30 mg/kg SOC-1 via oral gavage for 10 days. Body weights were recorded daily. Data from this experiment are presented in FIG. 5, which shows weight gain over the 10 dosing days of the experiment. Rats treated with 30 mg/kg SOC-1 had significantly inhibited weight gain over the 10 treatment days.

### REFERENCES

[1] Wojnicki, F. H. et al. (2006) Effects of baclofen on operant performance for food pellets and vegetable shortening after a history of binge-type behavior in non-food deprived rats. Pharmacol Biochem Behav 84 (2), 197-206.
[2] Nasser, J. A. et al. (2008) Use of an Operant Task to Estimate Food Reinforcement in Adult Humans With and Without BED. Obesity (Silver Spring, Md.) 16 (8), 1816-1820.
[3] Hu, F. B. and Malik, V. S. (2010) Sugar-sweetened beverages and risk of obesity and type 2 diabetes: Epidemiologic evidence. Physiol Behav 100 (1), 47-54.

It will be understood that the invention disclosed and defined in this specification extends to all alternative combinations of two or more of the individual features mentioned or evident from the text or drawings. All of these different combinations constitute various alternative aspects of the invention.

1. A method for causing weight loss in a subject, the method including a step of administering to the subject a compound of Formula (I), or a pharmaceutically acceptable salt, tautomer, N-oxide, solvate, polymorph and/or prodrug thereof: 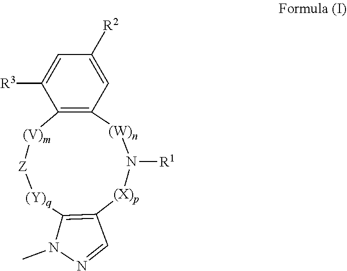
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

2. A method for managing weight in a subject, the method including a step of administering to the subject a compound of Formula (I), or a pharmaceutically acceptable salt, tautomer, N-oxide, solvate, polymorph and/or prodrug thereof: 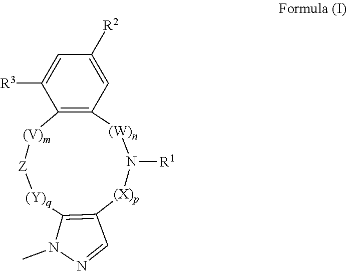
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

3. A method for suppressing an appetite for food in a subject, the method including a step of administering to the subject a compound of Formula (I), or a pharmaceutically acceptable salt, tautomer, N-oxide, solvate, polymorph and/or prodrug thereof: 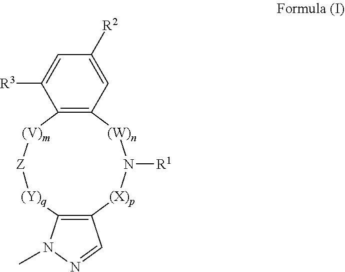
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

4. A method for controlling, reducing or minimising the consumption of food by a subject, the method including a step of administering to the subject a compound of Formula (I), or a pharmaceutically acceptable salt, tautomer, N-oxide, solvate, polymorph and/or prodrug thereof: 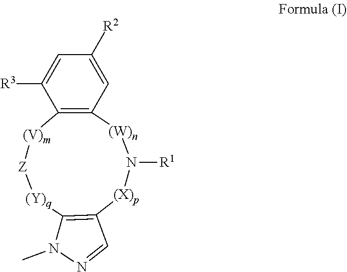
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

5. The method according to any one of the preceding claims, wherein the compound is of Formula (Ia): 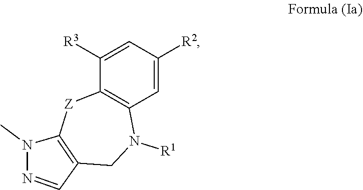
wherein:
Z is selected from: NH, O, S, S(O) or SO₂;
R¹ is selected from H or C(O)R⁴;
R² is selected from: H, a halogen, an optionally substituted C₁₋₅ alkyl, or an optionally substituted OC₁₋₅ alkyl;
R³ is selected from: H, a halogen, an optionally substituted C₁₋₅ alkyl, or an optionally substituted OC₁₋₅ alkyl; and
R⁴ is an optionally substituted C₁₋₅ alkyl,
or a salt, tautomer, N-oxide, solvate, polymorph and/or prodrug thereof.

6. The method according to any one of claims 1 to 4, wherein the compound is of Formula (Ib): 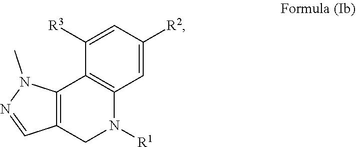
wherein:
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

7. The method according to any one of claims 1 to 4, wherein the compound is of Formula (Ic): 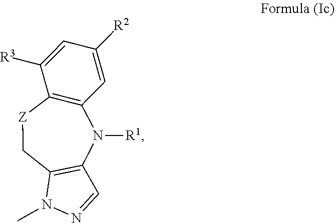
wherein:
Z is selected from: NH, O, S, S(O) or SO₂;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

8. The method according to any one of claims 1 to 4, wherein the compound is of Formula (Id): 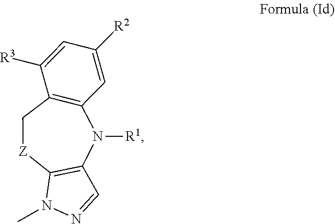
wherein:
Z is selected from: NH, O, S, S(O) or SO₂;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

9. The method according to any one of claims 1 to 4, wherein the compound is of Formula (Ie): 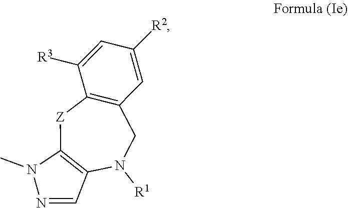
wherein:
Z is selected from: NH, O, S, S(O) or SO₂;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

10. The method according to any one of claims 1 to 9, wherein Z is NH.

11. The method according to any one of claims 1 to 9, wherein Z is O.

12. The method according to any one of claims 1 to 9, wherein Z is S.

13. The method according to any one of claims 1 to 9, wherein Z is S(O).

14. The method according to any one of claims 1 to 9, wherein Z is SO₂.

15. The method according to any one of claims 1 to 14, wherein R¹ is hydrogen.

16. The method according to any one of claims 1 to 14, wherein R¹ is C(O)R⁴.

17. The method according to any one of claim 1 to 14 or 16, wherein R⁴ is an optionally substituted C₁₋₅ alkyl selected from any one of: methyl, ethyl, propyl, isopropyl, n-butyl, sec-butyl, tert-butyl, n-pentyl, neopentyl, iso-pentyl and tert-pentyl.

18. The method according to any one of claim 1 to 14, 16 or 17, wherein R⁴ is an optionally substituted methyl.

19. The method according to any one of claims 1 to 18, wherein R² is hydrogen.

20. The method according to any one of claims 1 to 18, wherein R² is a hydroxyl group.

21. The method according to any one of claims 1 to 18, wherein R² is a halogen.

22. The method according to any one of claim 1 to 18 or 21, wherein R² is fluorine.

23. The method according to any one of claim 1 to 18 or 21, wherein R² is chlorine.

24. The method according to any one of claims 1 to 18, wherein R² is an optionally substituted C₁₋₅ alkyl.

25. The method according to any one of claim 1 to 18 or 24, wherein R² is an optionally substituted methyl.

26. The method according to any one of claims 1 to 18, wherein R² is an optionally substituted OC₁₋₅ alkyl.

27. The method according to any one of claim 1 to 18 or 26, wherein R² is an optionally substituted methoxy group.

28. The method according to any one of claims 1 to 27, wherein R³ is hydrogen.

29. The method according to any one of claims 1 to 27, wherein R³ is a hydroxyl group.

30. The method according to any one of claims 1 to 27, wherein R³ is a halogen.

31. The method according to any one of claim 1 to 27 or 30, wherein R³ is fluorine.

32. The method according to any one of claim 1 to 27 or 30, wherein R³ is chlorine.

33. The method according to any one of claims 1 to 27, wherein R³ is an optionally substituted C₁₋₅ alkyl.

34. The method according to any one of claim 1 to 27 or 33, wherein R³ is an optionally substituted methyl.

35. The method according to any one of claims 1 to 27, wherein R³ is an optionally substituted OC₁₋₅ alkyl.

36. The method according to any one of claim 1 to 27 or 35, wherein R³ is an optionally substituted methoxy group.

37. The method according to any one of claims 1 to 4, wherein Z is NH.

38. The method according to any one of claims 1 to 4, wherein R¹ is hydrogen.

39. The method according to any one of claims 1 to 4, wherein R² is hydrogen.

40. The method according to any one of claims 1 to 4, wherein R³ is hydrogen.

41. The method according to any one of claims 1 to 4, wherein the compound is selected from any one of: 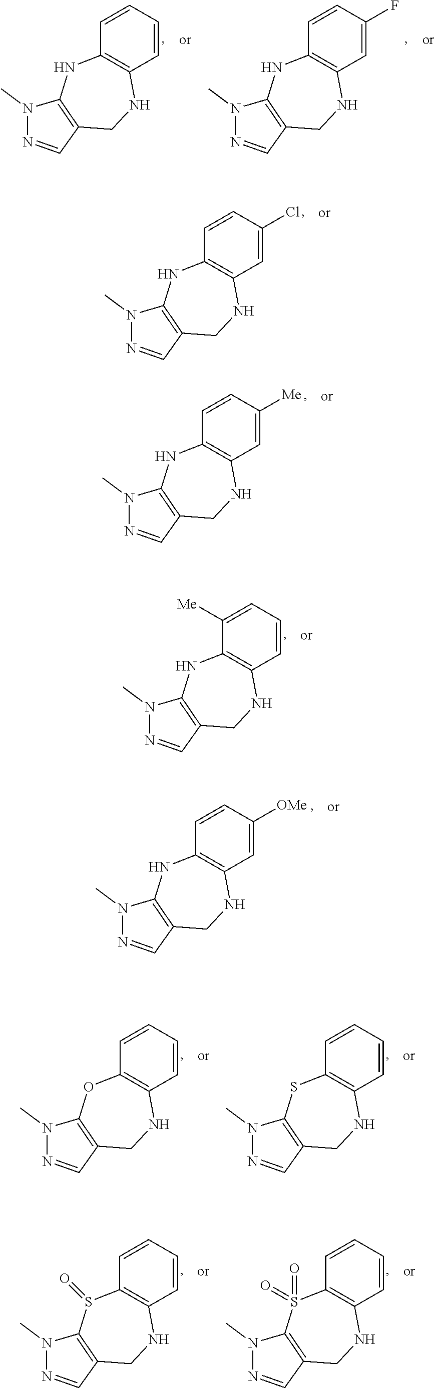 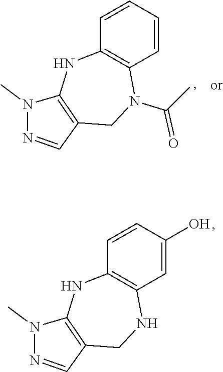
or a pharmaceutically acceptable salt, tautomer, N-oxide, solvate, polymorph and/or prodrug thereof.

42. The method according to claim any one of claims 1 to 4, wherein the compound is 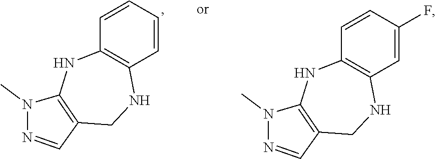 [SMILES: Cn1ncc2c1Nc1ccccc1NC2 ; Cn1ncc2c1Nc1ccc(F)cc1NC2]
or a pharmaceutically acceptable salt, tautomer, N-oxide, solvate, polymorph and/or prodrug thereof.

43. The method according to any one of claims 1 to 4, wherein the compound, or a pharmaceutically acceptable salt, tautomer, N-oxide, solvate, polymorph and/or prodrug thereof is

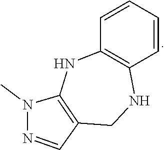

44. The method according to any one of claims 1 to 4, wherein the compound is a pharmaceutically acceptable salt of

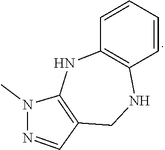

45. The method according to claim 44, wherein the compound is a pharmaceutically acceptable HCl salt of

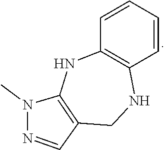

46. The method according to any one of claims 1 to 45, wherein the subject is obese.

47. The method according to any one of claims 1 to 45, wherein the subject is overweight.

48. The method according to any one of claims 1 to 45, wherein the subject is at risk of becoming overweight.

49. Use of a compound of Formula (I), or a pharmaceutically acceptable salt, tautomer, N-oxide, solvate, polymorph and/or prodrug thereof, in the manufacture of a medicament for causing weight loss in a subject: 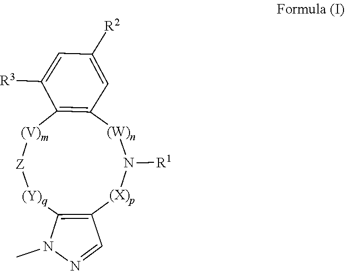
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

50. Use of a compound of Formula (I), or a pharmaceutically acceptable salt, tautomer, N-oxide, solvate, polymorph and/or prodrug thereof, in the manufacture of a medicament for managing weight in a subject: 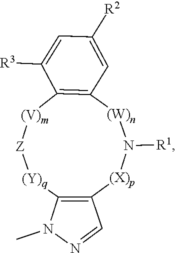
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

51. Use of a compound of Formula (I), or a pharmaceutically acceptable salt, tautomer, N-oxide, solvate, polymorph and/or prodrug thereof, in the manufacture of a medicament for suppressing an appetite for food in a subject: 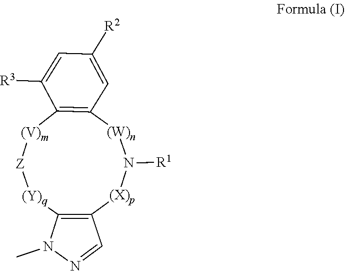
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

52. Use of a compound of Formula (I), or a pharmaceutically acceptable salt, tautomer, N-oxide, solvate, polymorph and/or prodrug thereof, in the manufacture of a medicament for reducing the consumption of food by a subject: 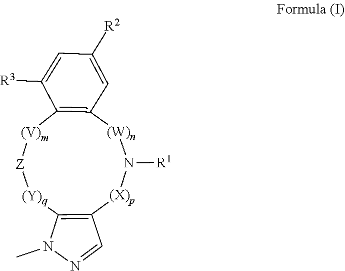
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

53. The use according to any one of claims 49 to 52, wherein the compound is of Formula (Ia): 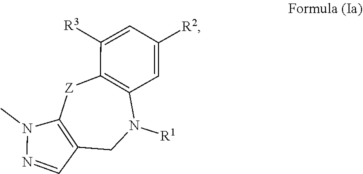
wherein:
Z is selected from: NH, O, S, S(O) or SO₂;
R¹ is selected from H or C(O)R⁴;
R² is selected from: H, a halogen, an optionally substituted C₁₋₅ alkyl, or an optionally substituted OC₁₋₅ alkyl;
R³ is selected from: H, a halogen, an optionally substituted C₁₋₅ alkyl, or an optionally substituted OC₁₋₅ alkyl; and
R⁴ is an optionally substituted C₁₋₅ alkyl,
or a pharmaceutically acceptable salt, tautomer, N-oxide, solvate, polymorph and/or prodrug thereof.

54. The use according to any one of claims 49 to 52, wherein the compound is of Formula (Ib): 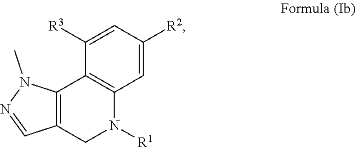
wherein:
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

55. The use according to any one of claims 49 to 52, wherein the compound is of Formula (Ic): 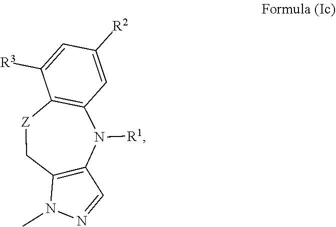
wherein:
Z is selected from: NH, O, S, S(O) or SO₂;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

56. The use according to any one of claims 49 to 52, wherein the compound is of Formula (Id): 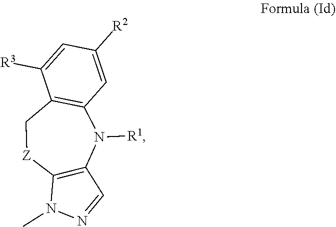
wherein:
Z is selected from: NH, O, S, S(O) or SO₂;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

57. The use according to any one of claims 49 to 52, wherein the compound is of Formula (Ie): 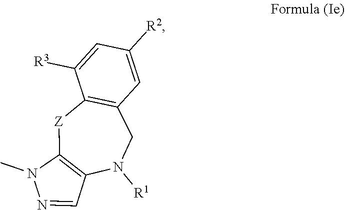
wherein:
Z is selected from: NH, O, S, S(O) or SO₂;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

58. The use according to any one of claims 49 to 57, wherein Z is NH.

59. The use according to any one of claims 49 to 57, wherein Z is O.

60. The use according to any one of claims 49 to 57, wherein Z is S.

61. The use according to any one of claims 49 to 57, wherein Z is S(O).

62. The use according to any one of claims 49 to 57, wherein Z is SO₂.

63. The use according to any one of claims 49 to 62, wherein R¹ is hydrogen.

64. The use according to any one of claims 49 to 62, wherein R¹ is C(O)R⁴.

65. The use according to any one of claim 49 to 62 or 64, wherein R⁴ is an optionally substituted C₁₋₅ alkyl selected from any one of: methyl, ethyl, propyl, isopropyl, n-butyl, sec-butyl, tert-butyl, n-pentyl, neopentyl, iso-pentyl and tert-pentyl.

66. The use according to any one of claim 49 to 62 or 64 or 65, wherein R⁴ is an optionally substituted methyl.

67. The use according to any one of claims 49 to 66, wherein R² is hydrogen.

68. The use according to any one of claims 49 to 66, wherein R² is a hydroxyl group.

69. The use according to any one of claims 49 to 66, wherein R² is a halogen.

70. The use according to any one of claim 49 to 66 or 69, wherein R² is fluorine.

71. The use according to any one of claim 49 to 66 or 69, wherein R² is chlorine.

72. The use according to any one of claims 49 to 66, wherein R² is an optionally substituted C₁₋₅ alkyl.

73. The use according to any one of claim 49 to 66 or 72, wherein R² is an optionally substituted methyl.

74. The use according to any one of claims 49 to 66, wherein R² is an optionally substituted OC₁₋₅ alkyl.

75. The use according to any one of claim 49 to 66 or 74, wherein R² is an optionally substituted methoxy group.

76. The use according to any one of claims 49 to 75, wherein R³ is hydrogen.

77. The use according to any one of claims 49 to 75, wherein R³ is a hydroxyl group.

78. The use according to any one of claims 49 to 75, wherein R³ is a halogen.

79. The use according to any one of claim 49 to 75 or 78, wherein R³ is fluorine.

80. The use according to any one of claim 49 to 75 or 78, wherein R³ is chlorine.

81. The use according to any one of claims 49 to 75, wherein R³ is an optionally substituted C₁₋₅ alkyl.

82. The use according to any one of claim 49 to 75 or 81, wherein R³ is an optionally substituted methyl.

83. The use according to any one of claims 49 to 75, wherein R³ is an optionally substituted OC₁₋₅ alkyl.

84. The use according to any one of claim 49 to 75 or 83, wherein R³ is an optionally substituted methoxy group.

85. The use according to any one of claims 49 to 52, wherein Z is NH.

86. The use according to any one of claims 49 to 52, wherein R¹ is hydrogen.

87. The use according to any one of claims 49 to 52, wherein R² is hydrogen.

88. The use according to any one of claims 49 to 52, wherein R³ is hydrogen.

89. The use according to any one of claims 49 to 52, wherein the compound is selected from any one of: 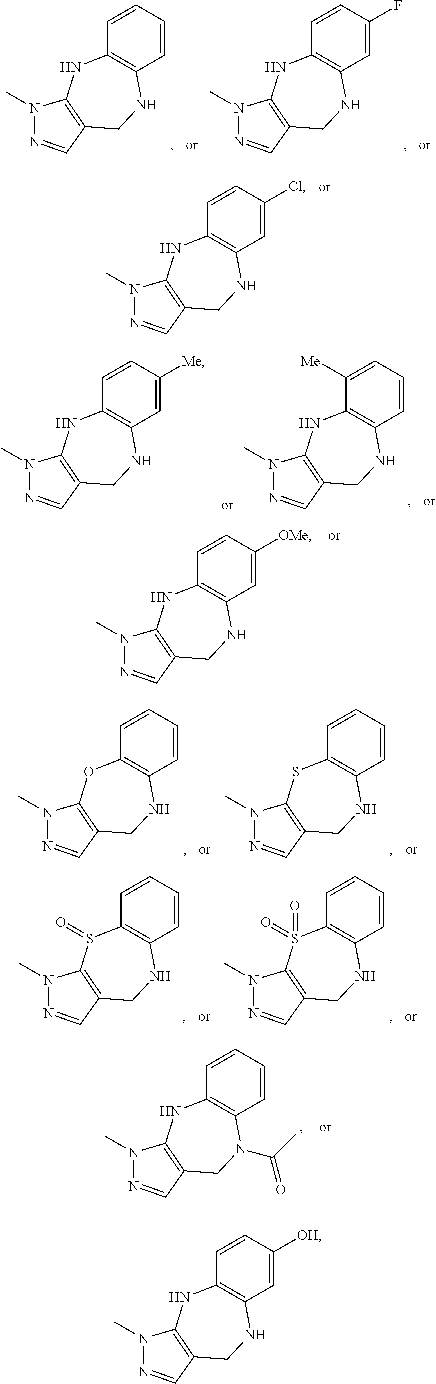
or a pharmaceutically acceptable salt, tautomer, N-oxide, solvate, polymorph and/or prodrug thereof.

90. The use according to any one of claims 49 to 52, wherein the compound is 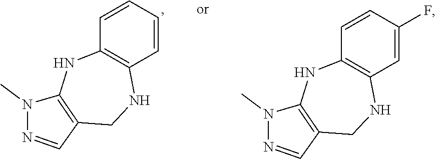 [SMILES: Cn1ncc2c1Nc1ccccc1NC2 ; Cn1ncc2c1Nc1ccc(F)cc1NC2]
or a pharmaceutically acceptable salt, tautomer, N-oxide, solvate, polymorph and/or prodrug thereof.

91. The use according to any one of claims 49 to 52, wherein the compound, or a pharmaceutically acceptable salt, tautomer, N-oxide, solvate, polymorph and/or prodrug thereof is

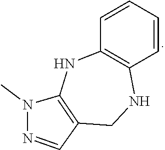

92. The use according to any one of claims 49 to 52, wherein the compound is a pharmaceutically acceptable salt of

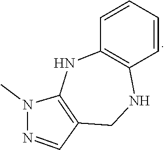

93. The use according to claim 92, wherein the compound is a pharmaceutically acceptable HCl salt of

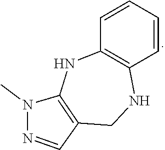

94. The use according to any one of claims 49 to 93, wherein the subject is obese.

95. The use according to any one of claims 49 to 93, wherein the subject is overweight.

96. The use according to any one of claims 49 to 93, wherein the subject is at risk of becoming overweight.

97. A compound of Formula (I), or a pharmaceutically acceptable salt, tautomer, N-oxide, solvate, polymorph and/or prodrug thereof, for use in a method for causing weight loss in a subject: 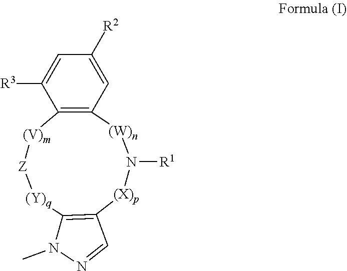
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

98. A compound of Formula (I), or a pharmaceutically acceptable salt, tautomer, N-oxide, solvate, polymorph and/or prodrug thereof, for use in a method for managing weight in a subject: 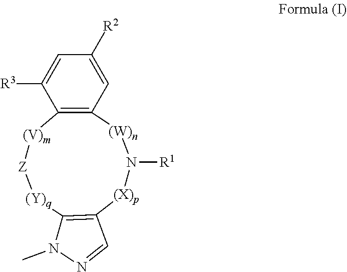
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

99. A compound of Formula (I), or a pharmaceutically acceptable salt, tautomer, N-oxide, solvate, polymorph and/or prodrug thereof, for use in a method for suppressing an appetite for food in a subject: 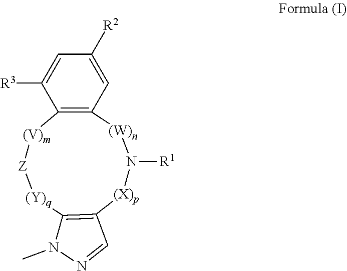
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

100. A compound of Formula (I), or a pharmaceutically acceptable salt, tautomer, N-oxide, solvate, polymorph and/or prodrug thereof, for use in a method for reducing the consumption of food by a subject: 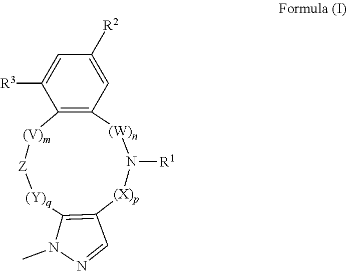
wherein:
V is NH, CH₂ or a direct bond;
W is NH, CH₂ or a direct bond;
X is NH, CH₂ or a direct bond;
Y is NH, CH₂ or a direct bond;
Z is selected from: NH, O, S, S(O), SO₂ or a direct bond;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl;
R⁴ is an optionally substituted C₁₋₅ alkyl;
m is 0 or 1;
n is 0 or 1;
p is 0 or 1; and
q is 0 or 1.

101. The compound, or a pharmaceutically acceptable salt, tautomer, N-oxide, solvate, polymorph and/or prodrug thereof, according to any one of claims 97 to 100, wherein the compound is of Formula (Ia): 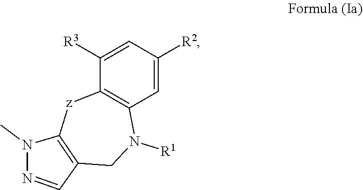
wherein:
Z is selected from: NH, O, S, S(O) or SO₂;
R¹ is selected from H or C(O)R⁴;
R² is selected from: H, a halogen, an optionally substituted C₁₋₅ alkyl, or an optionally substituted OC₁₋₅ alkyl;
R³ is selected from: H, a halogen, an optionally substituted C₁₋₅ alkyl, or an optionally substituted OC₁₋₅ alkyl; and
R⁴ is an optionally substituted C₁₋₅ alkyl,
or a pharmaceutically acceptable salt, tautomer, N-oxide, solvate, polymorph and/or prodrug thereof.

102. The compound, or a pharmaceutically acceptable salt, tautomer, N-oxide, solvate, polymorph and/or prodrug thereof, according to any one of claims 97 to 100, wherein the compound is of Formula (Ib): 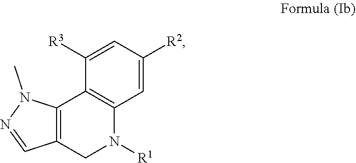
wherein:
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

103. The compound, or a pharmaceutically acceptable salt, tautomer, N-oxide, solvate, polymorph and/or prodrug thereof, according to any one of claims 97 to 100, wherein the compound is of Formula (Ic): 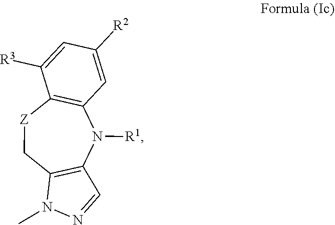
wherein:
Z is selected from: NH, O, S, S(O) or SO₂;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

104. The compound, or a pharmaceutically acceptable salt, tautomer, N-oxide, solvate, polymorph and/or prodrug thereof, according to any one of claims 97 to 100, wherein the compound is of Formula (Id): 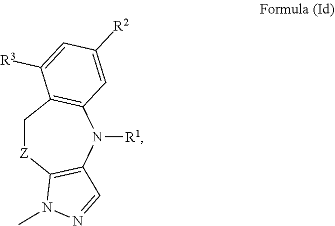
wherein:
Z is selected from: NH, O, S, S(O) or SO₂;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

105. The compound, or a pharmaceutically acceptable salt, tautomer, N-oxide, solvate, polymorph and/or prodrug thereof, according to any one of claims 97 to 100, wherein the compound is of Formula (Ie): 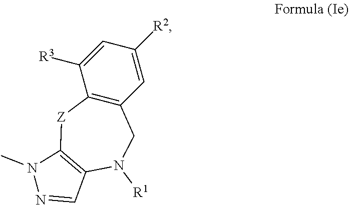
wherein:
Z is selected from: NH, O, S, S(O) or SO₂;
R¹ is selected from H or C(O)R⁴;
R² is selected from: H, OH, halogen, an optionally substituted C₁₋₅ alkyl or an optionally substituted OC₁₋₅ alkyl;
R³ is selected from: H, OH, halogen, an optionally substituted C₁₋₅ alkyl or an optionally substituted OC₁₋₅ alkyl; and
R⁴ is an optionally substituted C₁₋₅ alkyl,
or a salt, tautomer, N-oxide, solvate, polymorph and/or prodrug thereof.

106. The compound, or a pharmaceutically acceptable salt, tautomer, N-oxide, solvate, polymorph and/or prodrug thereof, according to any one of claims 97 to 105, wherein Z is NH.

107. The compound, or a pharmaceutically acceptable salt, tautomer, N-oxide, solvate, polymorph and/or prodrug thereof, according to any one of claims 97 to 105, wherein Z is O.

108. The compound, or a pharmaceutically acceptable salt, tautomer, N-oxide, solvate, polymorph and/or prodrug thereof, according to any one of claims 97 to 105, wherein Z is S.

109. The compound, or a pharmaceutically acceptable salt, tautomer, N-oxide, solvate, polymorph and/or prodrug thereof, according to any one of claims 97 to 105, wherein Z is S(O).

110. The compound, or a pharmaceutically acceptable salt, tautomer, N-oxide, solvate, polymorph and/or prodrug thereof, according to any one of claims 97 to 105, wherein Z is SO₂.

111. The compound, or a pharmaceutically acceptable salt, tautomer, N-oxide, solvate, polymorph and/or prodrug thereof, according to any one of claims 97 to 110, wherein R¹ is hydrogen.

112. The compound, or a pharmaceutically acceptable salt, tautomer, N-oxide, solvate, polymorph and/or prodrug thereof, according to any one of claims 97 to 110, wherein R¹ is C(O)R⁴.

113. The compound, or a pharmaceutically acceptable salt, tautomer, N-oxide, solvate, polymorph and/or prodrug thereof, according to any one of claim 97 to 110 or 112, wherein R⁴ is an optionally substituted C₁₋₅ alkyl selected from any one of: methyl, ethyl, propyl, isopropyl, n-butyl, sec-butyl, tert-butyl, n-pentyl, neopentyl, iso-pentyl and tert-pentyl.

114. The compound, or a pharmaceutically acceptable salt, tautomer, N-oxide, solvate, polymorph and/or prodrug thereof, according to any one of claim 97 to 110 or 112 or 113, wherein R⁴ is an optionally substituted methyl.

115. The compound, or a pharmaceutically acceptable salt, tautomer, N-oxide, solvate, polymorph and/or prodrug thereof, according to any one of claims 97 to 114, wherein R² is hydrogen.

116. The compound, or a pharmaceutically acceptable salt, tautomer, N-oxide, solvate, polymorph and/or prodrug thereof, according to any one of claims 97 to 114, wherein R² is a hydroxyl group.

117. The compound, or a pharmaceutically acceptable salt, tautomer, N-oxide, solvate, polymorph and/or prodrug thereof, according to any one of claims 97 to 114, wherein R² is a halogen.

118. The compound, or a pharmaceutically acceptable salt, tautomer, N-oxide, solvate, polymorph and/or prodrug thereof, according to any one of claim 97 to 114 or 117, wherein R² is fluorine.

119. The compound, or a pharmaceutically acceptable salt, tautomer, N-oxide, solvate, polymorph and/or prodrug thereof, according to any one of claim 97 to 114 or 117, wherein R² is chlorine.

120. The compound, or a pharmaceutically acceptable salt, tautomer, N-oxide, solvate, polymorph and/or prodrug thereof, according to any one of claims 97 to 114, wherein R² is an optionally substituted C₁₋₅ alkyl.

121. The compound, or a pharmaceutically acceptable salt, tautomer, N-oxide, solvate, polymorph and/or prodrug thereof, according to any one of claim 97 to 114 or 120, wherein R² is an optionally substituted methyl.

122. The compound, or a pharmaceutically acceptable salt, tautomer, N-oxide, solvate, polymorph and/or prodrug thereof, according to any one of claims 97 to 114, wherein R² is an optionally substituted OC₁₋₅ alkyl.

123. The compound, or a pharmaceutically acceptable salt, tautomer, N-oxide, solvate, polymorph and/or prodrug thereof, according to any one of claim 97 to 114 or 122, wherein R² is an optionally substituted methoxy group.

124. The compound, or a pharmaceutically acceptable salt, tautomer, N-oxide, solvate, polymorph and/or prodrug thereof, according to any one of claims 97 to 123, wherein R³ is hydrogen.

125. The compound, or a pharmaceutically acceptable salt, tautomer, N-oxide, solvate, polymorph and/or prodrug thereof, according to any one of claims 97 to 123, wherein R³ is a hydroxyl group.

126. The compound, or a pharmaceutically acceptable salt, tautomer, N-oxide, solvate, polymorph and/or prodrug thereof, according to any one of claims 97 to 123, wherein R³ is a halogen.

127. The compound, or a pharmaceutically acceptable salt, tautomer, N-oxide, solvate, polymorph and/or prodrug thereof, according to any one of claim 97 to 123 or 126, wherein R³ is fluorine.

128. The compound, or a pharmaceutically acceptable salt, tautomer, N-oxide, solvate, polymorph and/or prodrug thereof, according to any one of claim 97 to 123 or 126, wherein R³ is chlorine.

129. The compound, or a pharmaceutically acceptable salt, tautomer, N-oxide, solvate, polymorph and/or prodrug thereof, according to any one of claims 97 to 123, wherein R³ is an optionally substituted C₁₋₅ alkyl.

130. The compound, or a pharmaceutically acceptable salt, tautomer, N-oxide, solvate, polymorph and/or prodrug thereof, according to any one of claim 97 to 123 or 129, wherein R³ is an optionally substituted methyl.

131. The compound, or a pharmaceutically acceptable salt, tautomer, N-oxide, solvate, polymorph and/or prodrug thereof, according to any one of claims 97 to 123, wherein R³ is an optionally substituted OC₁₋₅ alkyl.

132. The compound, or a pharmaceutically acceptable salt, tautomer, N-oxide, solvate, polymorph and/or prodrug thereof, according to any one of claim 97 to 123 or 131, wherein R³ is an optionally substituted methoxy group.

133. The compound, or a pharmaceutically acceptable salt, tautomer, N-oxide, solvate, polymorph and/or prodrug thereof, according to any one of claims 97 to 100, wherein Z is NH.

134. The compound, or a pharmaceutically acceptable salt, tautomer, N-oxide, solvate, polymorph and/or prodrug thereof, according to any one of claims 97 to 100, wherein R¹ is hydrogen.

135. The compound, or a pharmaceutically acceptable salt, tautomer, N-oxide, solvate, polymorph and/or prodrug thereof, according to any one of claims 97 to 100, wherein R² is hydrogen.

136. The compound, or a pharmaceutically acceptable salt, tautomer, N-oxide, solvate, polymorph and/or prodrug thereof, according to any one of claims 97 to 100, wherein R³ is hydrogen.

137. The compound, or a pharmaceutically acceptable salt, tautomer, N-oxide, solvate, polymorph and/or prodrug thereof, according to any one of claims 97 to 100, wherein the compound is selected from any one of: 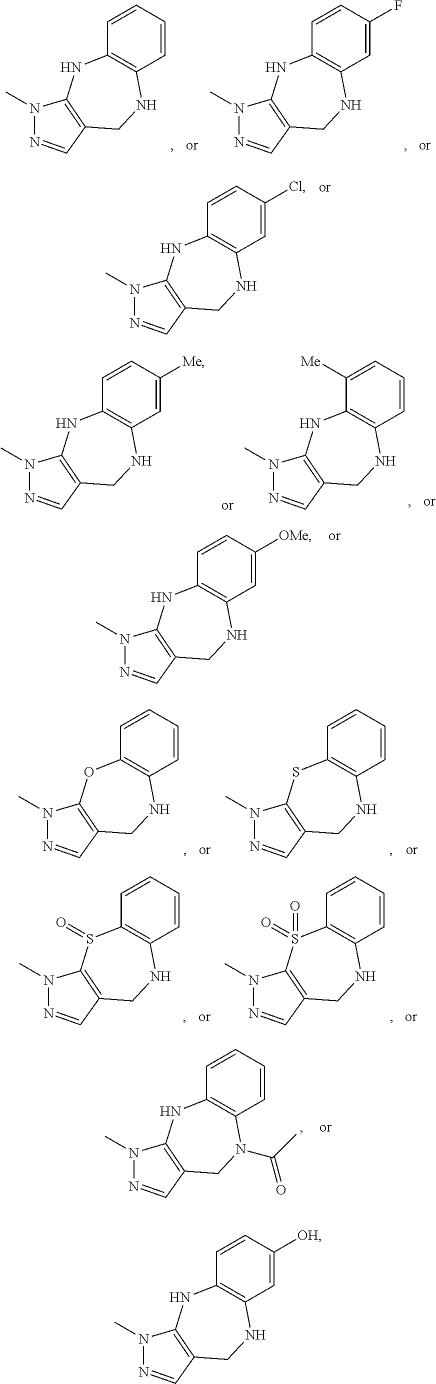
or a pharmaceutically acceptable salt, tautomer, N-oxide, solvate, polymorph and/or prodrug thereof.

138. The compound, or a pharmaceutically acceptable salt, tautomer, N-oxide, solvate, polymorph and/or prodrug thereof, according to any one of claims 97 to 100, wherein the compound is 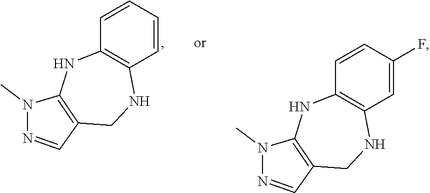 [SMILES: Cn1ncc2c1Nc1ccccc1NC2 ; Cn1ncc2c1Nc1ccc(F)cc1NC2]
or a pharmaceutically acceptable salt, tautomer, N-oxide, solvate, polymorph and/or prodrug thereof.

139. The compound, or a pharmaceutically acceptable salt, tautomer, N-oxide, solvate, polymorph and/or prodrug thereof, according to any one of claims 97 to 100, wherein the compound, or a pharmaceutically acceptable salt, tautomer, N-oxide, solvate, polymorph and/or prodrug thereof, is

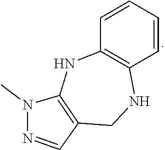

140. The compound, or a pharmaceutically acceptable salt, tautomer, N-oxide, solvate, polymorph and/or prodrug thereof, according to any one of claims 97 to 100, wherein the compound is a pharmaceutically acceptable salt of

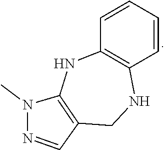

141. The compound, or a pharmaceutically acceptable salt, tautomer, N-oxide, solvate, polymorph and/or prodrug thereof, according to claim 140, wherein the compound is a pharmaceutically acceptable HCl salt of

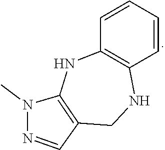

142. The compound, or a pharmaceutically acceptable salt, tautomer, N-oxide, solvate, polymorph and/or prodrug thereof, according to any one of claims 97 to 141, wherein the subject is obese.

143. The compound, or a pharmaceutically acceptable salt, tautomer, N-oxide, solvate, polymorph and/or prodrug thereof, according to any one of claims 97 to 141, wherein the subject is overweight.

144. The compound, or a pharmaceutically acceptable salt, tautomer, N-oxide, solvate, polymorph and/or prodrug thereof, according to any one of claims 97 to 141, wherein the subject is at risk of becoming overweight.
